(19) United States
(12) Patent Application Publication
(10) Pub. No.: US 2005/0032787 A1
(43) Pub. Date: Feb. 10, 2005

(54) Pheny (alkyl)carboxylic acid derivatives and dionic phenylalkylheterocyclic derivatives and their use as medicines with serum glucose and/or serum lipid lowering activity

(76) Inventors: Fabio Giannessi, Roma (IT); Tassoni Emanuela, Roma (IT); Dell'Uomo Natalina, Roma (IT); Brunetti Tiziana, Roma (IT); Tinti Maria Ornella, Roma (IT); Pessotto Pompeo, Roma (IT); Arduini Arduino, Roma (IT)

Correspondence Address: NIXON & VANDERHYE, PC, 1100 N GLEBE ROAD, 8TH FLOOR, ARLINGTON, VA, 22201-4714, US

(21) Appl. No.: 10/501,135

(22) PCT Filed: Jan. 13, 2003
(86) PCT No.: PCT/IT03/00007

(30) Foreign Application Priority Data
Jan. 15, 2002 (IT) RM2002A000016

(51) Int. Cl. A61K 31/54; A61K 31/537; A61K 31/495; A61K 31/42; A61K 31/415; A61K 31/195; A61K 31/19

(52) U.S. Cl. 514/227.5; 514/277; 514/408; 514/237.5; 514/252.12; 514/365; 514/374; 514/396; 514/570; 514/567

(57) ABSTRACT

Formula (I) compounds are described: Where the groups are as defined here below, and their use as medinies, particularly as serum glucose and serum lipid lowering agents. Said medicines are useful for the prophylaxis and treatment of diabetes, particularly type 2, and its complications, Syndrome X, the various forms of insulin resistance, and hyperlipidaemias, and present reduced side effects, and, particularly, reduced or no liver toxicity.

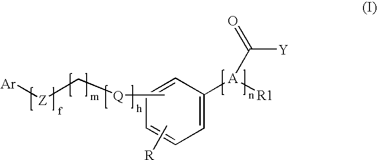

The invention described herein relates to phenyl(alkyl)carboxylic acid derivatives and dionic phenylalkyl-heterocyclic derivatives and to their use as medicines, particularly with serum glucose and/or serum lipid lowering activity.

### BACKGROUND OF THE INVENTION

Diabetes is a widespread disease throughout the world and is associated with major clinical complications including macrovascular (atherosclerosis) and microvascular (retinopathy, nephropathy and neuropathy) damage. Such complications are inevitable consequences of the disease and constitute a serious threat to the subject's life and well-being. Diabetes is associated with various abnormalities such as obesity, hypertension and hyperlipidaemia. Various clinical forms of diabetic disease are known, the most common being type 2 and type 1 diabetes. Type 2 diabetes is characterised by reduced sensitivity to the action of insulin (insulin resistance) and gives rise to an increase in actual insulin levels in the body in an attempt to compensate for this deficiency and to a consequent increase in glucose levels. Numerous reports have confirmed the involvement of insulin resistance in many disease conditions in addition to type 2 diabetes itself, such as dyslipidaemia, obesity, arterial hypertension and certain macro-vascular and microvascular manifestations characteristic of diabetes. The combination of insulin resistance and obesity, hypertension and dyslipidaemia is known as Syndrome X.

Drugs used for many years such as the biguanidines and sulphonylurea drugs are available on the market for the treatment of type 2 diabetes. In the case of the biguanidines (the best known of which is metformin) the mechanism of action is still unclear and the efficacy would not appear to afford satisfactory cover throughout all the hours of the night. Sulphonylurea drugs promote the secretion of insulin by the β-cells and may present episodes of hypoglycaemia as a possible side effect.

Drugs recently introduced onto the market are the thiazolidinediones, i.e. insulin-sensitising antidiabetic compounds such as troglitazone (J. Med. Chem., 1989, 32, 421-428), pioglitazone (Arzneim. Forsch/Drug Res., 1990, 40 (1), 37-42), and rosiglitazone (Bioorg. Med. Chem. Lett., 1994, 4, 1181-1184) which are capable of reducing hyperglycaemia, diabetic hyperlipidaemia and insulin levels. These compounds are high-affinity synthetic ligands of PPARγ (J. Biol. Chem., 1995, 270, 12953-12956).

Peroxisome proliferator activated receptors (PPARs) are receptors belonging to the superfamily of nuclear receptors whose function is to control the expression of genes involved in carbohydrate and lipid metabolism (J. Med. Chem., 2000, 43, 527-550). Various subtypes of PPARs have been identified: PPARγ, PPARα and PPARβ (also known as PPAR δ). The gamma isoform (PPARγ) is involved in the regulation of the differentiation of adipocyles and in energy homeostasis, whereas the alpha isoform (PPARα) controls fatty acid oxidation resulting in modulation of the levels of free fatty acids in plasma. In structure-activity relationship studies aimed at identifying new molecules endowed with potential antidiabetic action, a correspondence has been confirmed between PPARγ activation and serum glucose lowering activity (J. Med. Chem., 1996, 39, 665-668; J. Med. Chem., 1998, 41, 5020-5036; 5037-5054; 5055-5069). The insulin-sensitising action would appear to be related, as far as this first series of compounds is concerned, to the fatty acid recruitment action regulated by activated PPARγ which is thought to lead to an improvement in the insulin resistance of the tissues, enhancing serum glucose levels and lowering insulin levels. (Diabetes, 1998, 47, 507-514).

The side effects already observed with troglitazone and feared also in the case of the other compounds of this class are:: severe liver toxicity (which caused the withdrawal of troglitazione from the US market), increased cholesterol, weight gain and oedema.

In recent years molecules with a mixed profile, i.e. ligands of PPARγ and PPARα, have emerged (KRP 297, Diabetes, 1998, 47, 1841-1847; DRF 2725, Diabetes, 2001, 50, suppl.2, A108; AZ 242, Diabetes, 2001, 50, suppl. 2, A121-A122). These compounds are potentially capable of exerting a good measure of control of diabetic disease, while presenting a serum glucose and serum lipid lowering action with fewer side effects typical of the first series of compounds in the thiazolidinedione class, consisting exclusively of PPARγ ligands.

Not all the scientific community, however, agrees with this line of thinking. Recent studies on new-generation compounds, whether thiazolidinedione derivatives or otherwise (MC555, J. Biol. Chem., 1998, Vol. 273 (49), 32679-32684; NC2100 Diabetes, 2000, 49, 759-767, YM440, Metabolism, 2000, 49, 411-417), in gene transactivation tests, in-vitro glucose uptake experiments with muscle tissue and in-vivo experiments in transgenic animals with deficient PPARγ expression, have led to the hypothesis that there is no direct relationship between PPARγ activation and the serum glucose and serum lipid lowering activity of these compounds (Toxicology Letters, 2001, 120, 9-19). This may indicate that the serum glucose lowering activity of these molecules is not necessarily related to PPARγ activation and that these compounds may be capable of modulating carbohydrate and lipid metabolism through interaction with other biochemical targets. This is confirmed by the work of investigators who have opted for the use of in-vivo screening in diabetic animals (db/db mice, ob/ob mice) and for in-vitro/in-vivo tests (L6 cells), (J. Med. Chem., 1998, 41, 4556-4566) in order to identify possible insulin-sensitising agents which are not necessarily good PPAR ligands. These experiments have led to the selection of compounds still being investigated with promising antidiabetic activity in animal models (DRF 2189, J. Med. Chem., 1998, 41, 1619-1630; JTT-501, J. Med. Chem., 1998, 41, 1927-1933).

In conclusion, then, since the first compounds belonging to the thiazolidinedione class have proved to be associated with substantial hepatotoxic and other side effects, probably related to their PPARγ activity, the scientific community would now appear to be oriented towards the search for new compounds with a different mechanism of action which induce a similar or better effect on insulin sensitivity and glucose homeostasis without toxic side effects (J. Med. Chem., 2001, 44, 2601-2611).

### SUMMARY OF THEE INVENTION

It has now been found that compounds with formula (I) have been reported as being active as serum glucose and serum lipid lowering agents and are endowed with low toxicity and are therefore useful as medicines, particularly for the treatment of hyperlipidaemias and hyperglycaemias.

The preferred applications are the prophylaxis and treatment of diabetes, particularly type 2 and its complications, Syndrome X, the various forms of insulin resistance and hyperlipidaemias.

The object of the invention described herein are formula (I) compounds:

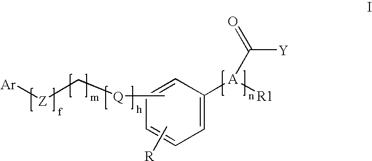

where:

A is CH; alkanylilidene with 2 to 4 carbon atoms, particularly CH₂—CH; alkenylilidene with 2 to 4 carbon atoms, particularly CH═C;

Ar is monocyclic, bicyclic or tricyclic C₆-C₁₀ aryl or heteroaryl, containing one or more heteroatoms selected from the group consisting of nitrogen, oxygen and sulphur, possibly substituted by halogens, NO₂, OH, C₁-C₄ alkyl and alkoxy, said alkyl and alkoxy possibly substituted by at least one halogen; monocyclic, bicyclic or tricyclic arylalkyl or heteroarylalkyl containing one or more heteroatoms selected from the group consisting of nitrogen, oxygen and sulphur, where the alkyl residue contains from 1 to 3 carbon atoms, said arylalkyl or heteroarylallyl possibly substituted by halogens, NO₂, OH, C₁-C₄ alkyl and alkoxy, said alkyl and alkoxy possibly substituted by at least one halogen;

f is the number 0 or 1;

h is the number 0 or 1;

m is a whole number from 0 to 3;

n is the number 0 or 1 and if n is 0, R₁ is absent, and COY is directly bound to benzene);

Q and Z, which may be the same or different, are selected from the group consisting of NH, O, S, NHC(O)O, NHC(O)NH, NHC(O)S, OC(O)NH, S(CO)NH, C(O)NH, and NHC(O);

R is selected from R₂, OR₂;

R₁ is selected from H, COW, SO₃—, OR₃, ═O, CN, NH₂, NHCO(C₆-C₁₀)Ar, where Ar may possibly be substituted by halogens, NO₂, OH, C₁-C₄ alkyl and alkoxy, said alkyl and alkoxy possibly substituted by at least one halogen;

R₂ is selected from H, straight or branched C₁-C₄ alkyl, possibly substituted by at least one halogen;

R₃ is selected from H, straight or branched C₁-C₄ alkyl, possibly substituted by at least one halogen, (C₆-C₁₀)ArCH₂, where Ar is possibly substituted by halogens, NO₂, OH, C₁-C₄ alkyl and alkoxy, said alkyl and alkoxy possibly substituted by at least one halogen;

W is selected from OH, OR₄, NH₂;

R₄ is straight or branched C₁-C₄ alkyl;

Y is selected from OH, OR₅, NH₂;

R₅ is straight or branched C₁-C₄ alkyl;

or A, COY and R₁ together form a cycle of the type:

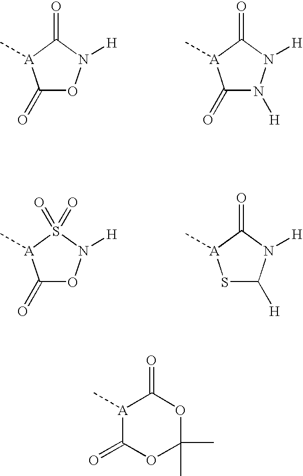

their pharmacologically acceptable salts, racemic mixtures, individual enantiomers, geometric isomers or stereoisomers, and tautomers.

A further object of the invention described herein is the use of said compounds as medicines for the treatment of hyperlipdaemias and hyperglycaemias, particularly for the treatment of type 2 diabetes and its complications, as well as pharmaceutical compositions containing said compounds as active ingredients.

These and other objects will be described in detail, also with the aid of examples.

### DETAILED DESCRIPTION OF THE INVENTION

In the formula (I) compounds, what is meant by alkanylilidene with 2 to 4 carbon atoms are the groups —(CR₆R₇)ₚ—CR₈<, where R₆, R₇ and R₈ are hydrogen, methyl or ethyl, and p is a whole number from 1 to 3. What is meant by alkenylilidene with 2 to 4 carbon atoms are the groups —CR₉R₁₀═C<, —CR₉R₁₀—CR₁₁═C<, —CR₉═CR₁₀—CR₁₁<, —CH2—CH2—CH═C<—CH═CH—CH2—CH<, —CH═CH—CH═C<, —CH2—CH═CH—CH<, —CH═C═CH—CH<, —CH2—CH═C═C<, where R₉, R₁₀ and R₁₁, are hydrogen, methyl or ethyl. In all cases the symbol<identifies the bond of A with COY and R₁.

In the formula (I) compounds, a first group of preferred compounds consists of compounds in which Ar is a heteroaryl, preferably containing nitrogen as the heteroatom, e.g. indole, or pyridine, bound to the rest of the molecule via all the positions allowed; particularly preferred among these are the 1-indolyl and 1-pyridyl groups. In the context of this first group, preferably f is 0, m is 1 or 2, Q is oxygen, and R is hydrogen.

A second group of preferred compounds consists of compounds in which Ar is an aryl, possibly substituted by one or more atoms of halogen, alkyl, alkoxy or lower haloalkyl, preferably methyl, methoxy or trifluoromethyl, nitro, mono- or di-alkylamine. In the context of this second group, preferably f is 0, m is 0, 1 or 2, Q is oxygen or HNC(O)O, and R is hydrogen.

Particularly preferred are the compounds where R₁ is COW.

Even more preferred are the following compounds:
i. Diethyl 4-[2-(1-indolyl)ethoxy]benzylidenemalonate
ii. Diethyl 4-[2-(1-indolyl)ethoxy]benzylmalonate
iii. Dimethyl 4-[2-(1-indolyl)ethoxy]benzylidenemalonate
iv. Dimethyl 4-[2-(1-indolyl)ethoxy]benzylmalonate
v. 4-[2-(1-indolyl)ethoxy]benzylmalonic acid
vi. Methyl (2S)-amino-2-[4-[12-(1-indolyl)ethoxy]phenyl]-acetate
vii. Methyl 4-[2-(1-indolyl)ethoxy]benzoate
viii. Methyl 3-[4-[2-(1-indolyl)ethoxy]phenyl]propanoate
ix. Methyl 2-[4-[2-(1-indolyl)ethoxy]phenyl]acetate
x. Methyl 2-sulpho-2-[4-[2-(1-indolyl)ethoxy]phenyl]acetate sodium salt
xi. Methyl (S)-2-benzoylamino-2-[4-[2-(1-indolyl)ethoxy]-phenyl]acetate
xii. Methyl 2-hydroxy-3-[4-[2-(1-indolyl)ethoxy]phenyl]-propanoate
xiii. Dimethyl 4-[2-[4-(dimethylamino)phenyl]ethoxy]benzylmalonate
xiv. Methyl 3-[4-[2-(1-indolyl)ethoxy]phenyl]-2-cyano-propenoate
xv. Methyl 3-[4-[2-(1-indolyl)ethoxy]phenyl]-2-cyano-propanoate
xvi. Dimethyl 4-[2-(3-indolyl)ethoxy]benzylidenemalonate
xvii. Dimethyl 4-[2-(1-naphthyl)ethoxy]benzylmalonate
xviii. Dimethyl 4-[2-(2-pyridyl)ethoxy]benzymalonate
xix. Dimethyl 4-[2-(4-chlorophenyl)ethoxy]benzylmalonate
xx. 5-[4-[2-(4-chlorophenyl)ethoxy]phenylmethylene]-thiazolidine-2,4-dione
xxi. 5-[4-[2-(4-chlorophenyl)ethoxy]phenylmethyl]thiazolidine-2,4-dione
xxii. Dimethyl 3-[2-(4-chlorophenyl)ethoxy]benzylmalonate
xxiii. Dimethyl 3-[2-(phenyl)ethoxy]benzylmalonate
xxiv. Dimethyl 3-[N-(4-trifluoromethylbenzyl)carbamoyl]-4-methoxybenzylmalonate
xxv. Dimethyl 4-methoxy-3-[2-(4-chlorophenyl)ethoxy]benzylmalonate
xxvi. Dimethyl 3-(2-phenylethoxy)-4-methoxy benzylmalonate
xxvii. Dimethyl 4-[2-(4-methoxyphenyl)ethoxy]benzyl-malonate
xxviii. Dimethyl 4-[3-(4-methoxyphenyl)propyloxy]benzylmalonate
xxix. Dimethyl 4-[2-(2-naphthyl)ethoxy]benzylmalonate
xxx. (2S)-2-benzoylamino-3-[4-[(4-methoxybenzyl)-carbamoyl]oxyphenyl]ethyl propanoate
xxxi. Dimethyl 4-[[(4-methoxybenzyl)carbamoyl]oxy]benzyl-malonate
xxxii. Dimethyl 4-[[(4-trifluorotolyl)carbamoyl]oxy]benzyl-malonate
xxxii. Dimethyl 4-[[(2,4-dichlorophenyl)carbamoyl]oxy]benzylmalonate
xxxv. Dimethyl 4-[[(4-chlorophenyl)carbamoyl]oxy]benzyl-malonate
xxxv. Dimethyl 4-[2-(pyridinio)ethoxy]benzylmalonate methanesulphonate
xxxvi. Dimethyl 4-[[(4-nitrophenyl)carbamoyl]oxy]benzyl-malonate
xxxvii. Dimethyl 3-[[(4-methoxybenzyl)carbamoyl]oxy]benzylmalonate
xxvii. Dimethyl 3-[[(4-butylphenyl)carbamoyl]oxy]benzyl-malonate
xxxix. Dimethyl 4-[[(4-butylphenyl)carbamoyl]oxy]benzyl-malonate
xl. Dimethyl 3-[[(4-chlorophenyl)carbamoyl]oxy]benzyl-malonate
xli. (Z)-2-ethoxy-3-[4-[2-(4-chlorophenyl)ethoxy]phenyl]ethyl propenoate
xlii. (E)-2-ethoxy-3-[4-[2-(4-chloro-phenyl)ethoxy]-phenyl]ethyl propenoate
xliii. (R,S)-2-ethoxy-3-[4-[2-(phenyl)ethoxy]phenyl]ethyl propanoate
xliv. (R,S)-2-ethoxy-3-[4-[2-(4-chloro-phenyl)ethoxy]-phenyl]methyl propanoate
xlv. Dimethyl 4-[2-(2,3-dimethyl-1-indolyl)ethoxy]benzyl-malonate

The formula compounds are prepared using the reactions described in methods A-H.

In the case of formula (I) compounds in which A is akenylilidene, R₁=COW, CN and Y=OH, OR₅, NH₂, or R₁ together with COY and A forms a cycle as indicated in formula (I) above, method A described here below can be used, as exemplified by A=—CH═C<.

Method A:

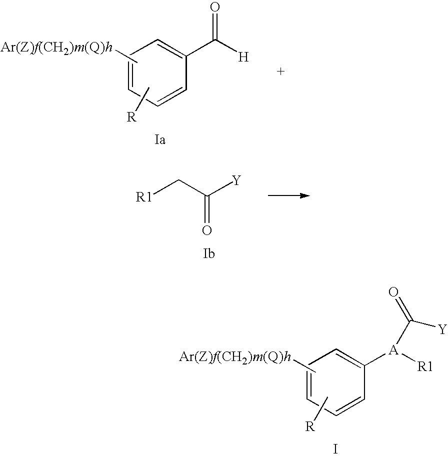

Unless otherwise specified, the meanings of the various symbols are intended to coincide with those indicated in the general formula.

The compounds of general formula I can be synthesised according to the diagram described above starting from compounds of general formula Ia and formula Ib in aprotic solvents such as toluene, refluxed with Dean-Stark, for time periods ranging from 5 to 24 hours, preferably 18 hours, in the presence, as a catalyst, of a salt of an organic base with an organic acid, such as piperidine acetate, normally used in Knovenagel reactions, or in aprotic dipolar solvents such as DMF (Synthetic Communications, 2000, 30 (4), 713-726), possibly in the presence of an organic base such as piperidine, at a temperature ranging from 20 to 100° C., preferably 80° C., for reaction times ranging from 1 hour to 3 days, preferably 2 days.

In the case of formula (I) compounds in which Q is selected from NH, O, S, NHC(O)S, and NHC(O)O, method B described here below can be used.

Method B:

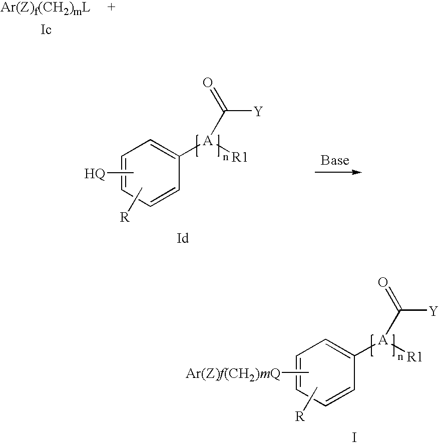

where L is an exit group such as MsO, TsO, Br, Cl, I

A, COY e R1 possono formare un ciclo=

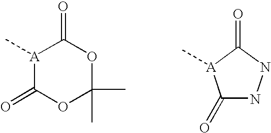

A, Coy and R1 may form a cycle=

Unless otherwise specified, the meanings of the various groups are intended to coincide with those indicated in formula (I) above.

The general formula I compounds can be synthesised according to the diagram described above starting from compounds of general formula Ic, Id, where L is an exit group, such as, for example, halogen, p-toluenesulphonate and methanesulphonate. The reaction is conducted in aprotic solvents such as DMF, DMSO and THF, in the presence of a base such as K₂CO₃ or KOH, or hydrides of alkaline metals such as NaH, possibly in an inert atmosphere which can be maintained using gases such as N₂ and Ar. The reaction temperature can range from 0 to 120° C., preferably 30-100° C., and the reaction times from 1 to 48 hours, preferably 6 to 18 hours.

In the case of formula (a) compounds in which Q is selected from O, or S, method C described here below can be used.

Method C:

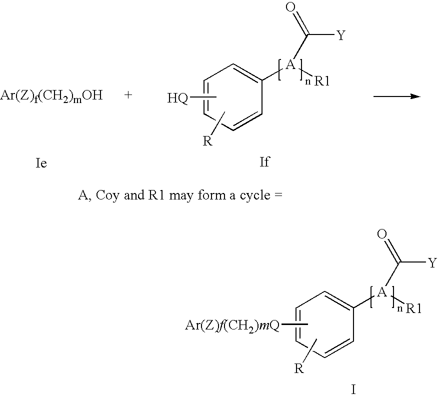

A, COY e R1 possono formare un ciclo=

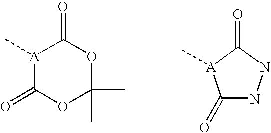

Unless otherwise specified, the meanings of the various groups are intended to coincide with those indicated in formula it) above. The general formula I compounds can be synthesised according to the diagram described above starting from compounds of general formula Ie, If, using as condensing agents triarylphosphine/dialkylazodicarboxylic esters such as PPH₃/DEAD and similar compounds that can be used in a ratio of 1 to 2 equivalents to the substrates, preferably 1.3-1.5 equivalents. The s reaction can be conducted in aprotic solvents such as THF, DME, CHCl₃ and the like, possibly in an inert atmosphere that can be maintained using gases such as N₂ and Ar. The reaction temperature can range from 0 to 60° C., preferably 20 to 40° C., and the reaction time from 3 hours to 6 days, preferably 18 hours to 3 days.

In the case of formula (I) compounds in which Q is selected from NHC(O)O, NHC(O)NH, NHC(O)S, OC(O)NH, or SC(O)NH, method D described here below can be used.

Method D:

Unless otherwise specified, the meanings of the various groups are intended to coincide with those indicated in formula (I) above, and X is —NCO when M is selected from OH, NH₂, SH, or X is OH, SH, NH₂ when M is NCO.

A, Coy and R1 may form a cycle=

A, COY e R1 possono formare un ciclo=

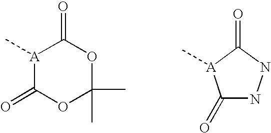

The general formula (I) compounds can be synthesised according to the diagram described above starting from compounds of general formula Ig, Ih, if M or X is an NCO group, in aprotic solvents such as CH₃CN, THF, CHCl₃ and the like, possibly in the presence, as a catalyst, of an organic base such as triethylamine, possibly in an inert atmosphere maintained with gases such as N₂ and Ar. The reaction temperature can range from 0 to 40° C., preferably 25° C., and the reaction time from 1 to 48 hours, preferably 18 hours.

In the case of formula (I) compounds in which Q is selected from NHC(O) or C(O)NH, method E described here below can be used.

Method E:

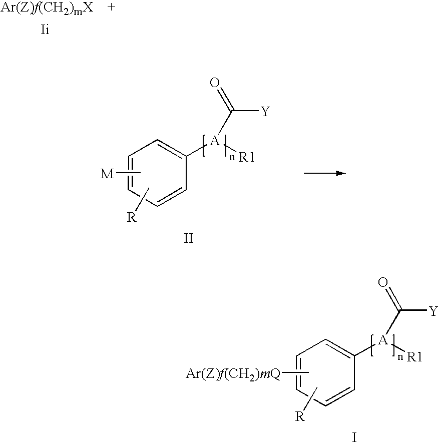

Unless otherwise specified, the meanings of the various groups are intended to coincide with those indicated in formula (I) above, and X is COOH when M is NH₂, and X is NH₂ when M is COOH.

The general formula (I) compounds can be synthesised according to the diagram described above starting from compounds of general formula Ii, Il when X or M is a COOH group, using condensing agents such as diethylphosphorocyanidate, EEDQ, DCC oo CDI and the like, in a ratio of 1-3 equivalents to the substrates, preferably 1-1.5 equivalents, conducting the reaction in organic solvents such as DMF, CH₃CN, CHCl₃, THF and the like, at a temperature ranging from 20 to 80° C., preferably 25° C., for reaction times ranging from 18 hours to 3 days, preferably 24 hours. The synthesis can also be conducted by derivatising the acid as acid halogenide and then effecting the condensation in the presence of a proton acceptor such as triethylamine, in conditions similar to those described above.

In the case of formula (I) compounds in which Ar is an aromatic heterocycle, method F described here below can be used, as exemplified by the pyridinium group.

Method F

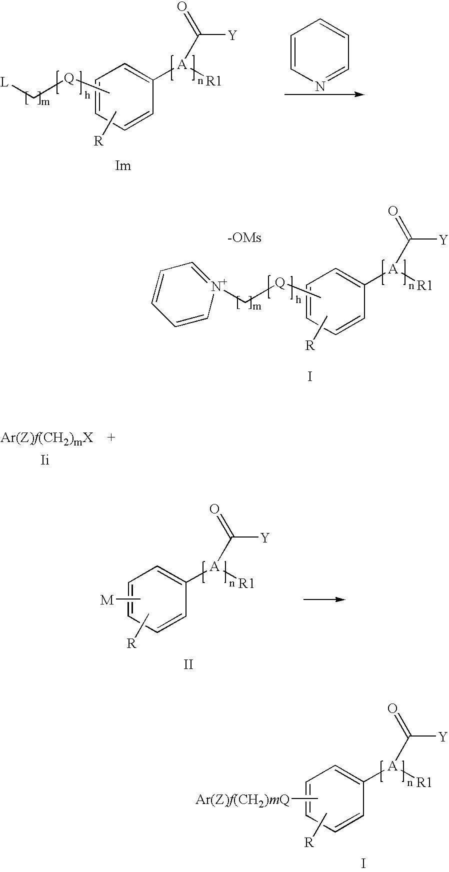

Unless otherwise specified, the meanings of the various groups are intended to coincide with those indicated in formula (a) above, and L is an exit group such as MsO, TsO, Br, Cl, or I; m is a whole number from 1 to 3.

The general formula (I) compounds can be synthesised starting from compounds of general formula Im according to the diagram described above, where L is an exit group such as, for example, halogen, p-toluenesulphonate and methanesulphonate. The reaction is conducted using the same conditions as described in method B.

In the case of formula (I) compounds in which Z takes on the meanings described in the general formula with the exclusion of NH, method G described here below can be used.

Method G:

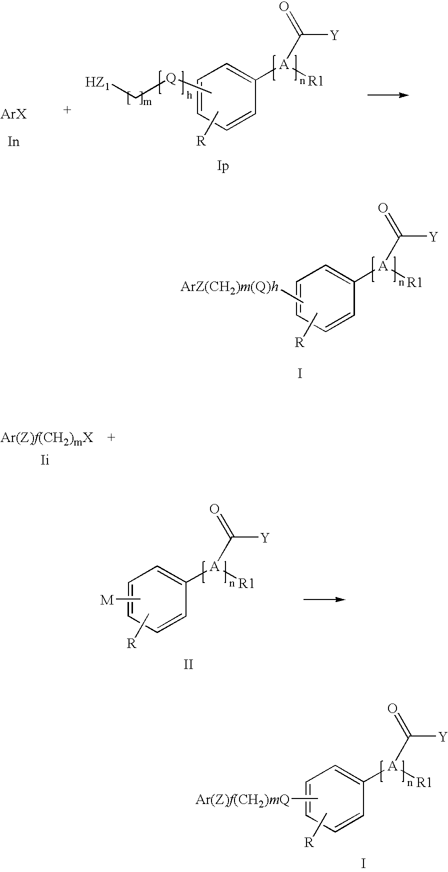

Unless otherwise specified, the meanings of the various groups are intended to coincide with those indicated in formula (I) above, and X is selected from NCO, COOH, OC(O)Cl, SC(O)Cl when Z₁ is selected from O, S, NH, or X is selected from OH, SH when Z₁ is O, or X is NH₂ when Z₁ is COOH.

The general formula (I) compounds can be synthesised starting from compounds of general formula In, Ip according to the diagram described above, when X or Z1 is a COOH group, and X or Z₁ is an O or N group, using the reaction conditions described in method E. When X is an NCO group and Z₁ is an O, N or S group, the reaction can be conducted in the conditions described in method D*. When X is an OH or SH group and Z₁ is an O group the reaction can be conducted as described in method C*. When X is an OC(O)Cl or SC(O)Cl group and Z₁ is an N group, the reaction is conducted in organic solvents such as CHCl₃, THF and the like, using a base such as triethylamine as the proton acceptor, at a temperature ranging from 0 to 60° C., preferably 25° C., for reaction times ranging from 2 to 24 hours, preferably 18 hours.
*In questi casi A, COY e R1 possono formare un ciclo=

*In these cases, A, Coy and R1 may form a cycle=

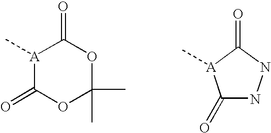

In the case of formula (a) compounds in which R₁=OR₃ and A=CH═C, method H described here below can be used.

Metodo H:

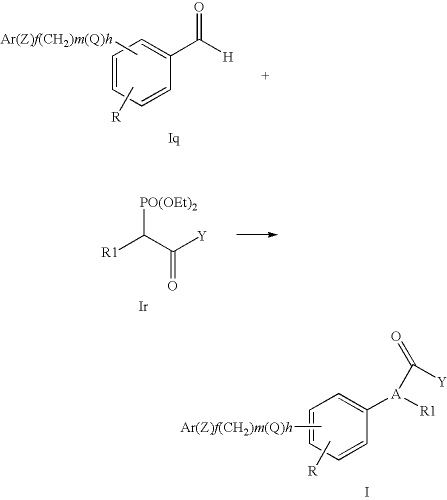

Unless otherwise specified, the meanings of the various groups are intended to coincide with those indicated in the general formula.

The general formula I compounds can be synthesised starting from compounds of general formula Iq and formula Ir (the latter obtained as described in Tetrahedron, 1992, 48 (19), 3991-4004), in aprotic solvents such as THF, in the presence of an inorganic base such as alcaline metal hydrides, preferably NaH, at a temperature ranging from 20 to 100° C., preferably ambient temperature, for reaction times ranging from 1 to 48 hours, preferably 20 hours.

In the case of formula (I) compounds in which A is alkanylilidene, these can be prepared from the corresponding formula (I) compounds where A is alkenylilidene.

Saturated compounds of general formula I can be obtained by reduction of the unsaturated compounds by catalytic hydrogenation in the presence of H₂, at a pressure ranging from atmospheric pressure to 60 psi, preferably 50 psi, and with catalysts such as metals supported on C, such as Pd/C, in percentages ranging from 1 to 20%, preferably 10%. The amount of catalyst used may fall within a range from 1 to 100% w/w, usually 10% w/w, in protic or aprotic solvents such as MeOH, dioxane and THF, preferably MeOH, for reaction times ranging from 18 hours to 3 days, preferably 24 hours. The reduction can also be conducted by means of hydrides such as NaBH₄ in organic solvents such as MeOH for reaction times ranging from 1 to 24 hours, preferably 2 hours, with a reaction temperature ranging from 0 to 80° C., preferably 25° C. An additional reduction method consists in the use of alkaline metals such as Mg in protic solvents such as MeOH, EtOH and the like at a temperature ranging from 20 to 40° C., preferably 25° C., for reaction times ranging from 2 to 24 hours, preferably 6 hours.

Unless otherwise indicated, the starting compounds are commercially available or can be prepared according to conventional methods, following the guidelines provided in the examples. The following examples further illustrate the invention.

### EXAMPLE 1

### Preparation of diethyl 4-[2-(1-indolyl)ethoxy]benzylidenemalonate (ST1445)

### Preparation of the intermediate product 1-(2-hydroxy-ethyl)indole

The intermediate product, reported in J. Med. Chem., 1998, 41/10, 1619-1639, was prepared according to the procedure described therein except for the duration of the reaction time (30 hours instead of 30 minutes), starting from indole (5.00 g, 42.7 mmol), KOH (3.60 g, 64.1 mmol) and from 2-bromoethanol (6.40 g, 51.3 mmol) in 50 mL of anhydrous DMSO, at T=25-30° C., to give 5.00 g of oily product (yield=73%).

### Preparation of the intermediate product 1-(2-methylsulphonyloxyethyl)indole

To a solution of 1-(2-hydroxyethyl)indole (1.00 g, 6.20 mmol), in 25 mL of anhydrous dichloromethane were added anhydrous pyridine (736 mg, 9.30 mmol) and, dropwise, methanesulphonyl chloride (1.06 g, 9.30 mmol). The reaction was left to stir at T=50° C. for 2 hours. After this time period the mixture was evaporated in vacuo and the residue dissolved in ethyl acetate (50 mL) and washed with H₂O (50 mL). The organic solution separated from the aqueous solution was washed with a solution of HCl 0.1N (2×50 mL) and with H₂O (2×50 mL). The organic solution was dried on anhydrous Na₂SO₄ and evaporated, and the residue was triturated with 100 mL of hexane to give 1.10 g of solid product after filtration (yield=74%). Melting point (Mp)=decomposes at 75° C.; TLC: silica gel, eluent AcOEt:hexane 3:7, Frontal ratio (Fr)=0.61; ¹H NMR (CDCl₃, 300 MHz) δ 7.62 (d, 1H), 7.38 (d, 1H), 7.22 (m, 2H), 7.18 (m, 2H), 6.57 (d, 1H), 4.50 (m, 4H), 2.60 (s, 3H); Elemental Analysis (E.A.) conforms for C₁₁ H₁₃ N O₃ S.

### Preparation of the intermediate product 4-[2-(1-indolyl)ethoxy]benzaldehyde

The intermediate product, reported in J. Med. Chem. 1998, 41(10), 1619-1639, was prepared with a different synthesis procedure, starting from the intermediate product 1-(2-methanesulphonyloxyethyl)indole (1.40 g, 5.85 mmol) and from 4-hydroxybenzaldehyde (880 mg 6.86 mmol) with NaH (190 mg, 7.87 mmol) in 30 mL of anhydrous DMF. The reaction mixture was left under continual stirring at a temperature of 80° C. for 18 hours. At the end of this time period H₂O (150 mL) was added to the mixture and the product was extracted with ethyl acetate (3×150 mL). The organic extracts collected were dried on anhydrous Na₂SO₄ and the solvent evaporated in vacuo to obtain 1.50 g of product (yield=96%).

### Preparation of diethyl 4-[2-(1-indolyl)ethoxy]benzylidenemalonate (ST1445)

### Method A

To a solution of 4-[2-(1-indolyl)ethoxy]benzaldehyde (1.40 g, 5.28 mmol) and diethylmalonate (845 mg, 5.28 mmol) in 15 mL of anhydrous toluene were added AcOH (47.2 mg, 0.79 mmol) and piperidine (66.9 mg, 0.79 mmol). The reaction mixture was left to reflux with Dean-Stark for 7 hours. After this time period the mixture was dried and the crude reaction product was purified by silica gel chromatography using AcOEt:hexane 3:7 as the eluent to give 1.50 g of oily product (yield=70%); TLC: silica gel, eluent AcOEt:hexane 3:7, Frontal ratio (Fr)=0.66; ¹H NMR (CDCl₃, 300 MHz) δ 7.60 (m, 2H), 7.40 (m. 3H), 7.22 (d, 1H), 7.20 (d, 1H), 7.15 (t, 1H), 6.80 (d, 2H), 6.45 (d, 1H), 4.45 (t, 2H), 4.25 (m, 6H), 1.25 (m, 6H); HPLC: column Inertisil ODS-3 (5 μm) (250×4.6 mm), mobile phase CH₃CN:H₂O (70:30 v/v), pH=as is, T=30° C., flow rate=0.75 mL/min, 205 nm UV detector, retention time=19.47 min; Elemental Analysis (E.A.) conforms for C₂₄H₂₅NO₅.

### EXAMPLE 2

### Preparation of diethyl 4-[2-(1-indolyl)ethoxy]benzylmalonate (ST1446)

ST1445, obtained as described in example 1, (0.90 g, 2.20 mmol) was dissolved in 30 mL of dioxane and subjected to catalytic hydrogenation (60 psi) with 10% Pd/C (90 mg) for 48 hours at ambient temperature. After this time period the suspension was filtered on celite and the filtrate evaporated in vacuo. The crude product was purified by flash chromatography on silica gel, using AcOEt:hexane 2:8 as the eluent, to give 380 mg of oily product (yield=42%); TLC: silica gel, eluent AcOEt:hexane 3:7, Frontal ratio (Fr)=0.60; ¹H NMR (CDCl₃, 300 MHz) δ 7.60 (d, 1H), 7.30 (d, 1H), 7.18 (m, 2H), 7.00 (m, 3H), 6.70 (d, 2H), 6.45 (d, 1H), 4.42 (t, 2H), 4.20 (t, 2H), 4.05 (m, 4H) 3.45 (t, 1H) 3.05 (d, 2H), 1.15 (t, 6H); HPLC: column: Inertisil ODS-3 (5 μm) (250×4.6 mm), mobile phase CH₃CN:H₂O (70:30 v/v), pH=as is, T=30° C., flow rate=0.75 mL/min, 205 nm UV detector, retention time=19.16 min; Elemental Analysis (E.A.) conforms for C₂₄H₂₇NO₅.

### EXAMPLE 3

### Preparation of dimethyl 4-[2-(1-indolyl]ethoxy]benzylidenemalonate (ST1443)

Method B

To a suspension of NaH (360 mg, 15.0 mmol) in anhydrous DMF (70 mL) was added, under N₂ flow, a solution of dimethyl 4-hydroxybenzylidenemalonate (3.00 g, 12.5 mmol) in 15 mL of anhydrous DMF. After clarification of the reaction mixture (30 minutes) a solution of 1-(2-methanesulphonyloxyethyl)indole was added, prepared as described in example 1, (2.90 g, 12.5 mmol), in 15 mL of anhydrous DMF, and the reaction mixture was left to stir for 18 hours at 70° C. under N₂ flow. After this time period H₂O (300 mL) was added to the reaction and the product was extracted with ethyl acetate (3×100 mL). The organic solution was washed with H₂O and with a saturated solution of NaCl, dried on anhydrous Na₂SO₄ and evaporated dry in vacuo. The crude reaction product was purified by flash chromatography on silica gel using AcOEt:hexane 2:8 as the eluent to give 3.10 g of solid product (yield=65%). Melting point (Mp)=68-70° C.; TLC: silica gel, eluent AcOEt:hexane 3:7, Frontal ratio (Fr)=0.61; ¹H NMR (CDCl₃, 300 MHz) δ 7.65 (s, 1H), 7.62 (d, 1H), 7.40 (m, 3H), 7.20 (m, 3H), 6.82 (d, 2H), 6.50 (d, 1H), 4.50 (t, 2H), 4.30 (t, 2H), 3.80 (d, 6H); HPLC: column: Symmetry C18 (5 μm) (150×3.9 mm), mobile phase CH₃CN:KH₂PO₄ 50 mM (60:40 v/v), pH=3, T=30° C., flow rate=0.5 mL/min, 205 nm UV detector, retention time=12.75 min; Elemental Analysis (E.A.) conforms for C₂₂H₂₁NO₅.

### EXAMPLE 4

### Preparation of dimethyl 4-[2-(1-indolyl)ethoxy]benzylmalonate (ST1444)

ST1443, prepared as described in example 3, (1.50 g, 3.90 mmol), was dissolved in 45 mL of dioxane and subjected to catalytic hydrogenation (60 psi) with 10% Pd/C (750 mg) for 24 hours at ambient temperature. The suspension was filtered on celite and the filtrate was evaporated in vacuo to give an oily residue that was purified by silica gel chromatography using AcOEt:hexane 2:8 as the eluent to give 0.90 g of oily product (yield=60%); TLC: silica gel, eluent AcOEt:hexane 3:7, Frontal ratio (Fr)=0.63; ¹H NMR (CDCl₃, 300 MHz) δ 7.62 (d, 1H), 7.40 (d, 1H), 7.20 (m, 2H), 7.10 (2d, 3H), 6.80 (d, 2H), 6.50 (d, 1H), 4.50 (t, 2H), 4.25 (t, 2H), 3.70 (s, 6H), 3.60 (t, 1H), 3.15 (d, 2H); HPLC: column: Symmetry C18 (5 μm) (150×3.9 mm), mobile phase CH₃CN:KH₂PO₄ 50 mM (60:40 v/v), pH=3, T=30° C., flow rate=0.5 mL/min, 205 nm UV detector, retention time=13.15 min; Elemental Analysis (E.A.) conforms for C₂₂H₂₃NO₅.

### EXAMPLE 5

### Preparation of 4-[2-(1-indolyl)ethoxy]benzylmalonic acid (ST1467)

To a solution of ST1444, prepared as described in example 3, (0.95 g, 2.50 mmol), in methanol (10 mL) and THF (5 mL), was added NaOH 2N (3 mL) and the reaction was left to stir at ambient temperature for 24 hours. After this time period the reaction was evaporated in vacuo, water (10 mL) was added to the residue, and the solution was extracted with AcOEt (2×10 mL). The aqueous phase was acidified with HCl 1 N to pH=4 and the product was extracted with AcOEt (2×10 mL). The organic extracts were dried on anhydrous Na₂SO₄ and evaporated in vacuo. The residue was redissolved in AcOEt and precipitated with hexane to give 250 mg of product (yield=28%); Melting point (Mp)=112-114° C. TLC: silica gel, eluent AcOEt:hexane 3:7, Frontal ratio (Fr)=0.28; ¹H NMR (CDCl₃, 300 MHz) δ 7.60 (d, 1H), 7.50 (d, 1H), 7.30 (d, 1H), 7.20 (t, 1H), 7.10 (m, 3H), 6.80 (d, 2H), 6.45 (d, 1H), 4.50 (t, 2H), 4.30 (t, 2H), 3.60 (t, 1H), 3.05 (d, 2H); HPLC: column: Symmetry C18 (5 μm) (150×3.9 mm), mobile phase CH₃CN:KH₂PO₄ 50 mM (55:45 v/v), pH=4, T=30° C., flow rate=0.5 mL/min, 205 nm UV detector, retention time=4.40 min; Elemental Analysis (E.A.) conforms for C₂₀H₁₉NO₅, KF=0.8% H₂O.

### EXAMPLE 6

### Preparation of methyl (2S)-amino-2-[4-[2-(1-indolyl)ethoxy]phenyl]acetate (ST1539)

### Preparation of the intermediate product 4-hydroxy-(2S)-α-phenylglycine hydrochloride methyl ester

To a solution of 4-hydroxy-(2S)-α-phenylglycine (5.00 g, 29.0 mmol) in MeOH (50 mL) was added SOCl₂ (7.20 g, 59.0 mmol). The reaction was left to stir at ambient temperature for 24 hours. The solvent was evaporated in vacuo and the residue triturated with diethyl ether to give 6.50 g of product as a white solid (yield=100%); TLC: silica gel, eluent AcOEt:hexane 5:5, Frontal ratio (Fr)=0.21; ¹H NMR (CDCl₃, 300 MHz) δ7.30 (d, 2H), 6.90 (d, 2H), 5.20 (s, 1H), 3.80 (s, 3H).

### Preparation of methyl (2S)-amino-2-[4-[2-(1-indolyl)ethoxy]-phenyl]acetate (ST1539)

The product was prepared as described in example 3 (method B) starting from 4-hydroxy (2S)-α-phenylglycine hydrochloride methyl ester (1.10 g, 5.00 mmol) and from 1-(2-methylsulphonyloxyethyl)indole, prepared as described in example 1 (1.20 g, 5.00 mol) in anhydrous DMF (50 mL), except for the amount of NaH (280 mg, 12.0 mmol), the reaction time (6 hours instead of 18 hours) and the eluent used in the purification by chromatography (AcOEt instead of AcOEt:hexane 2:8), to give 500 mg of oily product (yield=31%); [α]_{D}²⁰=−7° (c=0.1 in MeOH); TLC: silica gel, eluent AcOEt:MeOH 9:1, Frontal ratio (Fr)=0.51; ¹H NMR (CDCl₃, 300 MHz) δ 7.62 (d, 1H), 7.40 (d, 1H), 7.22 (m, 4H), 7.10 (t, 1H), 6.80 (d, 2H), 6.55 (d, 1H), 4.50 (s+t, 3H), 4.30 (t, 2H), 3.70 (s, 3H); HPLC: column: Symmetry C18 (5 μm) (250×4.6 mm), mobile phase CH₃CN:KH₂PO₄ 50 mM (60:40 v/v), pH=4.2, T=30° C., flow rate=0.75 mL/min, 205 nm UV detector, retention time=6.52 min; Elemental Analysis (E.A,) conforms for C₁₉H₂₀N₂O₃.

### EXAMPLE 7

### Preparation of methyl 4-[2-(1-indolyl)ethoxyi]benzoate (ST1671)

The product was prepared as described in example 3 (method B) from 1-(2-methanesulphonyloxyethyl)indole, prepared as described in example 1 (0.95 g, 3.90 mmol), methyl 4-hydroxybenzoate (600 mg, 3.90 mmol) and NaH (114 mg, 4.70 mmol), in anhydrous DMF (10 mL), except for the reaction time (24 hours instead of 18 hours) and the eluent used in the purification by chromatography (AcOEt:hexane 1:9 instead of 2:8). The still impure product obtained was purified by chromatography on Amberlyst A21 resin using AcOEt as the eluent to give 540 mg of product as a white solid (yield=47%); Melting point (Mp)=70-73° C., TLC: silica gel, eluent AcOEt:hexane 3:7, Frontal ratio (Fr)=0.48; ¹H NMR (CDCl₃, 300 MHz) δ 8.00 (d, 2H), 7.65 (d, 1H), 7.40 (d, 1H), 7.20 (m, 3H), 6.90 (d, 2H), 6.60 (d, 1H), 4.60 (t, 2H), 4.40 (t, 2H), 3.90 (s, 3H); HPLC: column: Symmetry (5 μm)-(250×4.6 mm), mobile phase CH₃CN:KH₂PO₄ 50 mM (60:40 v/v), pH=as is, T=30° C., flow rate=0.75 mL/min, 205 nm UV detector, retention time=24.66 min; Elemental Analysis (E.A.) conforms for C₁₈H₁₇NO₃.

### EXAMPLE 8

### Preparation of methyl 3-[4-[2-(1-indolyl)ethoxy]phenyl]-propanoate (ST1626)

The product was prepared as described in example 3 (method B) from 1-(2-methanesulphonyloxyethyl)indole, prepared as described in example 1, (1.10 g, 4.50 mmol), methyl 4-hydroxyphenylpropanoate (820 mg, 4.55 mmol) and NaH (142 mg, 5.90 mmol), except for the solvent (anhydrous acetonitrile (1.5 mL) instead of anhydrous DMF) and the eluent used in the purification by chromatography (AcOEt:hexane 1:9 instead of 2:8). The residue obtained was triturated further with hexane to eliminate traces of is solvent, to give 270 mg of product as a white solid (yield=19%); Melting point (Mp)=85° C., TLC: silica gel, eluent AcOEt:hexane 3:7, Frontal ratio (Fr)=0.49; ¹H NMR (CDCl₃, 300 MHz) δ7.62 (d, 1H), 7.40 (d, 1H), 7.20 (m, 3H),, 7.10 (d, 2H), 6.80 (d, 2H), 6.50 (d,1H), 4.50 (t, 2H), 4.30 (t, 2H), 3.82 (s, 3H), 2.90 (t, 2H), 2.60 (t, 2H); HPLC: column: Symmetry (5 μm)-(250×4.6 mm), mobile phase CH₃CN:H₂O (60:40 v/v), pH=as is, T=30° C., flow rate=0.75 mL/min, 205 nm UV detector, retention time=22.33 min; Elemental Analysis (E.A.) conforms for C₂₀H₂₁NO₃.

### EXAMPLE 9

### Preparation of methyl 2-[4-[2-(1-indolyl)ethoxy]phenyl]acetate (ST1627)

The product was prepared as described in example 3 (method B) from 1-(2-methanesulphonyloxyethyl)indole, prepared as described in example 1 (860 mg, 3.60 mmol), methyl 4-hydroxyphenylacetate (600 mg, 3.60 mmol) and NaH (112 mg, 4.70 mmol), except for the solvent (anhydrous acetonitrile (1.5 mL) instead of anhydrous DMF) and the eluent used in the purification by chromatography (AcOEt:hexane 1:9 instead of 2:8) to give 243 mg of product as a white solid (yield=22%); Melting point (Mp)=50-52° C., TLC: silica gel, eluent AcOEt:hexane 3:7, Frontal ratio (Fr)=0.46; ¹H NMR (CDCl₃, 300 MHz) δ7.62 (d, 1H), 7.40 (d, 1H), 7.20 (m, 5H), 6.80 (d, 2H), 6.55 (d,1H), 4.58 (t, 2H), 4.30 (t, 2H), 3.70 (s, 3H), 3.60 (s, 2H); HPLC: column: Symmetry (5 μm)-(250×4.6 mm), mobile phase CH₃CN:H₂O (60:40 v/v), pH=as is, T=30° C., flow rate=0.75 mL/min, 205 nm UV detector, retention time=17.38 min; Elemental Analysis (E.A.) conforms for C₁₉H₁₉NO₃.

### EXAMPLE 10

### Preparation of methyl 2-sulpho-2-[4-[2-(1-indolyl)ethoxy]-phenyl]acetate sodium salt (ST1706)

### Preparation of the intermediate product methyl 4-hydroxy-α-sulphophenylacetate sodium salt

The product was prepared from 4-hydroxy-α-sulphophenylacetic acid sodium salt monohydrate (2.00 g, 7.34 mmol) disolved in MeOH (44 mL) with the addition of SOCl₂ (1.75 g, 14.6 mmol). The reaction mixture was left at ambient temperature for 24 hours After evaporation of the solvent in vacuo the residue was treated with diethyl ether (3×50 mL). The still impure final residue was purified by flash chromatography on silica gel using CHCl₃:MeOH 8:2 as the eluent to give 1.25 g of oily product (yield=63.5%); ¹H NMR (D₂O, 300 MHz) δ7.30 (d, 2H), 6.80 (d, 2H), 4.95 (s, 1H), 3.65 (s, 3H); Elemental Analysis (E.A.) conforms for C₉H₁₀SO₆Na; KF=2.2% H₂O.

### Preparation of methyl 2-sulpho-2-[4-[2-(1-indolyl)ethoxy]-phenyl]acetate sodium salt (ST1706)

The product was prepared as described in example 3 (method B) starting from methyl 4-hydroxy-sulphophenylacetate sodium salt (1.10 g, 4.10 mmol), 1-(2-methanesulphonyloxyethyl)indole, prepared as described in example 1, (0.98 g, 4.10 mmol), and NaH (147.6 mg, 6.15 mmol) in 3.4 mL of anhydrous DMF, except for the reaction time and the temperature (3 hours instead of 18 hours, at 120° C. rather than at 80° C.). The dark semisolid was treated with diethyl ether (200 mL) and the crude solid obtained was purified by flash chromatography on silica gel using CHCl_{3:}MeOH 9:1 as the eluent to give 400 mg of solid product (yield=21.4%); Melting point (Mp)=253-258° C. (decomposes); TLC: silica gel, eluent CHCl₃:MeOH 7:3, Frontal ratio (Fr)=0.58; ¹H NMR (CD₃OD_{d4}, 300 MHz) δ7.55 (m, 4H), 7.25 (d, 1H), 7.18 (t, 1H), 7.00 (t, 1H) 6.80 (d, 2H), 6.42 (d, 1H), 4.85 (s, 1H), 4.50 (t, 2H), 4.30 (t, 2H), 3.70 (s, 3H); HPLC: column: Symmetry C18 (5 μm) (250×4.6 mm), mobile phase CH₃CN:KH₂PO₄ 50 mM (50:50 v/v), pH=3, T=30° C., flow rate=1 mL/min, 205 nm UV detector, retention time=6.07 min; Elemental Analysis (E.A.) conforms for C₁₉H₁₈NO₆NaS.

### EXAMPLE 11

### Preparation of methyl (S)-2-benzoylamino-2-[4-[2-(1-indolyl)-ethoxy]phenyl]acetate (ST1709)

### Preparation of the intermediate product methyl (S)-2-benzoylamino-2-(4-hydroxyphenyl)acetate

The product was prepared from 4-hydroxy-(2S)-α-phenylglycine methyl ester hydrochloride, prepared as described in example 6, (1.24 g, 5.70 mmol) dissolved in DMF (30 mL), adding TEA (1.15 g, 11.4 mmol) and benzoyl chloride (896 mg, 6.38 mmol) to the solution at 0° C. The reaction mixture was left at ambient temperature for 18 hours. After this time period H₂O (100 mL) was added to the reaction and the product was extracted with ethyl acetate (3×30 mL). The organic solution was washed with H₂O (2×40 mL), dried on anhydrous Na₂SO₄ and evaporated dry in vacuo, to give 1.29 g of solid product (yield=79%); Melting point (Mp)=152° C.; ¹H NMR (CDCl₃, 300 MHz)δ 7.90 (d, 2H), 7.50 (m, 3H), 7.20 (d, 2H), 6.80 (d, 2H), 5.70 (d, 1H), 3.80 (s, 3H).

### Preparation of methyl (2S)-benzoylamino-2-[4-[2-(1-indolyl)-ethoxy]phenyl]acetate (ST1709)

The product was prepared as described in example 3 (method B) starting from methyl (2S)-benzoylamino-2-(4-hydroxyphenyl)acetate (0.70 g, 2.50 mmol), 1-(2-methanesulphonyloxyethyl)indole, prepared as described in example 1 (0.58 g, 2.50 mmol) and NaH (72 mg, 3.00 mmol) for 24 hours (instead of 18 hours). In the processing CH₂Cl₂ was used for extraction of the product with water instead of ethyl acetate The chromatographic purification of the product was done using AcOEt:hexane 7:3 (instead of 2:8) as the eluent to give 530 mg of oily product Wield=50%); [α]_{D}²⁰=−2.6° (c=1% in CHCl₃); TLC: silica gel, eluent AcOEt:hexane 5:5, Frontal ratio (Fr)=0.65; ¹H NMR (CDCl₃, 300 MHz)δ7.80 (d, 2H), 7.60 (d, 1H), 7.55-7.10 (m, 9H), 6.82 (d, 2H), 6.50 (d, 1H), 5.70 (d, 1H), 4.50 (t, 2H), 4.22 (t, 2H), 3.75 (s, 3H); HPLC: column: Inertisil ODS-3 (5 μm) (250×4.6 mm), mobile phase CH₃CN:KH₂PO₄ 50 mM (65:35 v/v), pH=as is, T=30° C., flow rate=0.75 mL/min, 205 nm UV detector, retention time=13.57 min; Elemental Analysis (E.A.) conforms for C₂₆H₂₄N₂O₄, KF=1.5% H₂O.

### EXAMPLE 12

### Preparation of methyl 2-hydroxy-3-[4-[2-(1-indolyl)ethoxy]-phenyl]propanoate (ST1733)

### Preparation of the intermediate product methyl 2-hydroxy-3-(4-hydroxy)phenyl)propanoate

The product was prepared from D,L 3-(4-hydroxyphenyl)lactic acid hydrate (500 mg, 2.76 mmol) dissolved in MeOH (30 mL) with gaseous HCl to saturation. The reaction solution was left at ambient temperature for 4 hours. After evaporation of the solvent in vacuo the oily residue was re-dissolved with diethyl ether and the solvent evaporated in vacuo, repeating the operation 3 times (3×10 mL) to give 540 mg of oily product (yield=100%); ¹H NMR (CDCl₃, 300 MHz) δ7.10 (d, 2H), 6.90 (d, 2H), 5.00 (brs, 1H), 4.45 (t, 1H), 3.80 (s, 3H), 3.00 (dd, 2H).

### Preparation of methyl 2-hydroxy-3-[4-[2-(1-indolyl)ethoxy]-phenyl]propanoate (ST1733)

The product was prepared as described in example 3 (method B) starting from methyl 2-hydroxy-3-(4-hydroxyphenyl)propanoate (800 mg, 4.10 mmol) and 1-(2-methanesulphonyloxyethyl)indole, prepared as described in example 1 (970 mg, 4.10 mmol) and NaH (108 mg, 4.50 mmol) in 50 mL of anhydrous DMF, at 40° C. for 24 hours (instead of at 70° C. for 18 hours). In the processing the product was extracted with CH₂Cl₂ instead of ethyl acetate and the final residue was purified by chromatography using AcOEt:hexane 3:7 (instead of 2:8) as the eluent to give 270 mg of solid product (yield=18%); Melting point (Mp)=70-72° C.; TLC; silica gel, eluent AcOEt:hexane 3:7, Frontal ratio (Fr)=0.22; ¹H NMR (CDCl₃, 300 MHz) δ7.65 (d, 1H), 7.40 (d, 1H), 7.12 (m, 3H), 7.10 (d, 2H), 6.80 (d, 2H), 6.55 (d, 1H), 4.50 (t, 2H), 4.40 (brt, 1H), 4.22 (t, 2H), 3.80 (s, 3H), 3.00 (dq, 2H); HPLC: column: Inertisil ODS-3 (5 μm)-(250×4.6 mm), mobile phase CH₃CN:KH₂PO₄ 50 mM (65:35 v/v), pH=as is, T=30° C., flow rate=0.75 mL/min, 205 nm UV detector, retention time=9.39 min; Elemental Analysis (E.A.). conforms for C₂₀H₂₁NO₄.

### EXAMPLE 13

### Preparation of dimethyl 4-[2-[4-(dimethylamino)phenyl]-ethoxy]benzylmalonate (ST 1705)

### Preparation of the intermediate product 1-methanesulfonyloxy-2-[4-(dimethylamino)phenyl]ethyl

To a solution of 4-(dimethylamino)phenylethanol (500 mg, 3.02 mmol), in anhydrous dichloromethane (10 mL), were added TEA (336 mg, 3.33 mmol) and, dropwise, methanesulphonyl chloride (381 mg, 3.33 mmol) at 0° C. The reaction was left at ambient temperature for 18 hours. After this time period the mixture was evaporated in vacuo, the residue was extracted with AcOEt (100 mL) and the solution filtered. The organic solution was evaporated in vacuo to give 720 mg of oily product (yield=98%); ¹H NMR (CDCl₃, 300 MHz) δ 7.10 (d, 2H), 6.70 (d, 2H), 4.40 (t, 2H), 3.00 (m, 8H), 2.85 (s, 3H).

### Preparation of the intermediate product dimethyl 4-hydroxybenzylmalonate

The product was prepared from dimethyl 4-hydroxybenzylidenemalonate (5.00 g, 21.0 mmol) by catalytic hydrogenation with 10% Pd/C (500 mg) in MeOH, as described in the method in patent WO 94/13650 Heterocyclic derivatives and their use in pharmaceuticals, except for the duration of the reaction time (24 hours instead of 5 hours) and the pressure (50 psi instead of ambient pressure) to give 5.00 g of oily product (yield=99%); the analytical data resemble those reported in the literature described.

### Preparation of dimethyl 4-[2-[4-(dimethylamino)phenyl]-ethoxy]benzylmalonate (ST1705)

The product was prepared as described in example 3 (method B) starting from dimethyl 4-hydroxybenzylmalonate (708 mg, 2.97 mmol), 1-methanesulphonyloxy-2-[4-(dimethylamino)phenyl]ethyl (724 mg, 2.97 mmol) and NaH (71 mg, 2.97 mmol). The crude reaction product was purified by flash chromatography on silica gel using AcOEt:hexane 15:85 (instead of 2:8) as the eluent to give the oily product that was further purified by treatment with hexane to give 270 mg of product (yield=24%); TLC: silica gel, eluent AcOEt:hexane 4:6, Frontal ratio (Fr)=0.55; ¹H NMR (CDCl₃, 300 MHz) δ7.18 (d, 2H), 7.12 (d, 2H), 6.80 (d, 2H), 6.75 (m, 2H), 4.10 (t, 2H), 3.70 (s, 6H), 3.60 (t, 1H), 3.18 (d, 2H), 3.00 (t, 2H), 2.90 (s, 6H); HPLC: column: Symmetry C18 (5 μm) (250×4.6 mm), mobile phase CH₃CN:H₂O (65:35 v/v), pH=as is, T=30° C., flow rate=0.75 mL/min, 205 nm UV detector, retention time=19.13 min; Elemental Analysis (E.A.) conforms for C₂₂H₂₇NO₅.

### EXAMPLE 14

### Preparation of methyl 3-[4-[2-(1-indolyl)ethoxy]phenyl]-2-cyanopropenoate (ST1462)

### Preparation of the intermediate product methyl α-cyano-4-hydroxycinnamate

To a solution of α-cyano-4-hydroxycinnamic acid (20.0 g, 106 mmol) in MeOH (200 mL) was added SOCl₂ (24.9 g, 210 mmol). The reaction was left to stir at T=60° C. for 24 hours. The solvent was evaporated in vacuo and the residue triturated with diethyl ether to give 18.0 g of product as a pale yellow solid (yield=85%); TLC: silica gel, eluent AcOEt:hexane 3:7, Frontal ratio (Fr)=0.28; ¹H NMR (CDCl₃, 300 MHz) δ8.20 (s, 1H), 8.10 (d, 2H), 7.10 (d, 2H), 3.90 (s, 3H).

### Preparation of methyl 3-[4-[2-(1-indolyl)ethoxy]phenyl]-2-cyanopropenoate (ST1462)

Method C

To a solution of 1-(2-hydroxyethyl)indole, prepared as described in example 1, (1.00 g, 6.20 mmol) and methyl α-cyano-4-hydroxycinnamate (1.10 g, 5.60 mmol) in anhydrous THF (20 mL) were added DEAD (1.30 g, 7.3 mmol) and PPh₃ (1.90 g, 7.30 mmol). The solution was left to stir at ambient temperature for 5 days. The residue obtained after evaporation of the solvent in vacuo was purified by flash chromatography on SiO₂ gel using AcOEt:hexane 2:8 as the eluent to give 850 mg of solid product (yield=44%); Melting point (Mp)=142-144° C.; TLC: silica gel, eluent AcOEt:hexane 3:7, Frontal ratio (Fr)=0.38; ¹H NMR (CDCl₃, 300 MHz) δ 8.10 (s, 1H), 7.90 (d, 2H), 7.60 (d, 1H), 7.35 (d, 1H), 7.10 (m, 2H), 7.05 (t, 1H), 6.80 (d, 2H), 6.45 (d, 1H), 4.50 (t, 2H), 4.25 (t, 2H), 3.80 (s, 3H); HPLC: column: Symmetry C18 (5 μm)-(150×3.9 mm), mobile phase CH₃CN:H₂O (60:40 v/v), pH=as is, T=30° C., flow rate=0.5 mL/min, 205 nm UV detector, retention time=13.86 min; Elemental Analysis (E.A.) conforms for C₂₁H₁₈N₂O₃.

### EXAMPLE 15

### Preparation of methyl 3-[4-[2-(1-indolyl)ethoxy]phenyl]-2-cyanopropanoate (ST1499)

ST1462, re-prepared as described in example 14 (1.30 g, 3.70 mol), was dissolved in 60 mL of THF and subjected to catalytic hydrogenation (15 psi) with 10% Pd/C (130 mg) for 24 hours. The suspension was filtered on celite, the filtrate evaporated in vacuo and the residue purified by flash chromatography on SiO₂ gel, using AcOEt:hexane 3:7 as the eluent to give 620 mg of oily product (yield=48%); TLC: silica gel, eluent AcOEt:hexane 3:7, Frontal ratio (Fr)=0.42; ¹H NMR (CDCl₃, 300 MHz) δ7.62 (d, 1H), 7.40 (d, 1H), 7.20 (m, 5H), 6.80(d, 2H), 6.55 (d, 1H), 4.50(t, 2H), 4.30(t, 2H), 3.80 (s, 3H), 3.65 (t, 1H), 3.15 (m, 2H); HPLC: column: Symmetry C18 (5 μm)-(250×4.6 mm), mobile phase CH₃CN:H₂O (70:30 v/v), pH=as is, T=30° C., flow rate=0.75 mL/min, 205 nm UV detector, retention time=14.47 min; Elemental Analysis (E.A.) conforms for C₂₁H₂₀N₂O₃.

### EXAMPLE 16

### Preparation of dimethyl 4-[2-(3-indolyl)ethoxy]benzylidenemalonate (ST1474)

The product was prepared as described in example 14 (method C) starting from 3-(2-hydroxyethyl)indole, (2.50 g, 15.5 mmol), dimethyl 4-hydroxybenzylidenemalonate (3.30 g, 14.1 mmol), DEAD (3.20 g, 18.3 mmol) and PPh₃ (4.80 g, 18.3 mmol), except for the reaction time (4 days instead of 5 days) and the eluent used in the purification by chromatography (AcOEt:hexane 3:7 and isopropyl ether:hexane 6:4 instead of AcOEt:hexane 2:8) to give a solid residue which was crystallised with AcOEt and hexane to give 480 mg of product (yield=9.5%); Melting point (Mp)=105.7° C.; TLC: silica gel, eluent AcOEt:hexane 1:1, Frontal ratio (Fr)=0.65; ¹H NMR (CDCl₃, 300 MHz) δ8.00 (brs,1H), 7.65 (s, 1H), 7.61 (d, 1H), 7.40 (m, 3H), 7.20 (m, 3H), 6.85 (d, 2H), 4.25 (t, 2H), 3.82 (d, 6H), 3.22 (t, 2H); HPLC: column: Symmetry (5 μm) (150×3.9 mm), mobile phase CH₃CN:KH₂PO₄ 50 mM (50:50 v/v), pH=3, T=30° C., flow rate=0.5 mL/min, 205 nm UV detector, retention time=22.85 min; Elemental Analysis (E.A.) conforms for C₂₂H₂₁O₅.

### EXAMPLE 17

### Preparation of dimethyl 4-[2-(1-naphthyl)ethoxy]benzyl-malonate (ST1475)

The product was prepared as described in example 14 (method C) starting from 1-(2-hydroxyethyl)naphthalene (1.50 g, 8.70 mmol), dimethyl 4-hydroxybenzylmalonate, prepared as described in example 13, (1.90 g, 7.90 mmol), DEAD (1.90 g, 11.3 mmol) and, PPh₃ (2.90 g, 11.3 mmol), except for the reaction time (1 day instead of 5 days) to give 1.90 g of oily product after purification (yield=61%); TLC: silica gel, eluent AcOEt:hexane 2:8, Frontal ratio (Fr)=0.42; ¹H NMR (CDCl₃, 300 MHz) δ 8.10 (d, 1H), 7.90 (d, 1H), 7.70 (t, 1H), 7.47 (m, 2H), 7.42 (d, 2H), 7.10 (d, 2H) 6.80 (d, 2H), 4.25 (t, 2H), 3.62 (s, 6H), 3.60 (m, 3H), 3.20 (d, 2H); HPLC: column: Symmetry (5 μm) (150×3.9 mm), mobile phase CH₃CN:KH₂PO₄ 50 mM (55:45 v/v), pH=3, T=30° C., flow rate=0.7 mL/min, 205 nm UV detector, retention time=28.46 min; Elemental Analysis (E.A.) conforms for C₂₄H₂₄O₅.

### EXAMPLE 18

### Preparation of dimethyl 4-[2-(2-pyridyl)ethoxy]benzylmalonate (ST1476)

The product was prepared as described in example 14 (method C) starting from 2-(2-hydroxyethyl)pyridine (800 mg, 6.40 mmol), dimethyl 4-hydroxybenzylmalonate, prepared as described in example 13, (1.70 g, 6.90 mmol), DEAD (1.40 g, 8.00 mmol) and PPh₃ (2.10 g, 8.00 mmol), except for the reaction time (3 days instead of 5 days) and the eluent used in the purification by chromatography (AcOEt:hexane [3:7 instead of 2:8]) to give 850 mg of oily product (yield=38%); TLC: silica gel, eluente AcOEt:hexane 1:1, Frontal ratio (Fr)=0.36; ¹H NMR (CDCl₃, 300 MHz) δ 8.50 (d, 1H), 7.60 (td, 1H), 7.22 (d, 1H), 7.12 (m, 1H), 7.08 (d, 2H), 6.80 (d, 2H), 4.32 (t, 2H), 3.70 (s, 6H), 3.60 (t, 1H), 3.22 (t, 2H) 3.15 (d, 2H); HPLC: column: Symmetry (5 μm) (150×3.9 mm), mobile phase CH₃CN:KH₂PO₄ 50 mM (25:75 v/v), pH=3, T=30° C., flow rate=0.5 mL/min, 205 nm UV detector, retention time=11.71 min; Elemental Analysis (E.A.) conforms for C₁₉H₂₁NO₅, KF=3.14% H₂O.

### EXAMPLE 19

### Preparation of dimethyl 4-[2-(4-chlorophenyl)ethoxy]benzyl-malonate (ST1493)

The product was prepared as described in example 14 (method C) starting from 2-(4-chlorophenyl)ethanol (700 mg, 4.60 mmol), dimethyl 4-hydroxybenzylmalonate, prepared as described in example 13, (1.20 g, 5.00 mmol), DEAD (1.10 g, 5.90 mmol) and PPh3 (1.60 g, 5.90 mmol), except for the reaction time (3 days instead of 5 days) and the eluent used in the purification by chromatography (AcOEt:hexane [3:7 instead of 2:8]) to give 800 mg of oily product (yield=47%); TLC: silica gel, eluent AcOEt:hexane 3:7, Frontal ratio (Fr)=0.47; ¹H NMR (CDCl₃, 300 MHz) δ 7.22 (q, 4H), 7.11 (d, 2H), 6.80 (d, 2H), 4.20 (t, 2H), 3.70 (s, 6H), 3.6 (t, 1H), 3.15 (d, 2H) 3.05 (t, 2H); HPLC: column: Symmetry (5 μm) (150×3.9 mm), mobile phase CH₃CN:KH₂PO₄ 50 mM (55:45 v/v), pH=5.5, T=30° C., flow rate=1.0 mL/min, 205 nm UV detector, retention=23.42 min; Elemental Analysis (E.A.) conforms for C₂₀H₂₁CIO₅.

### EXAMPLE 20

### Preparation of 5-[4-[2-(4-chlorophenyl)ethoxy]phenylmethylene]thiazolidine-2,4-dione (ST1862)

### Preparation of the intermediate product 4-[2-(4-chloro-phenyl)ethoxy]benzaldehyde

The product was prepared as described in example 14 (method C) starting from 4-hydroxybenzaldehyde (2.00 g, 16.4 mmol), 2-(4-chlorophenyl)ethanol (2.80 g, 18.0 mmol), PPh₃ (5.57 g, 21.3 mmol) and DEAD (3.70 g, 21.3 mmol), except for the reaction time (one night instead of 5 days). 2.60 g of product were obtained after purification (yield=61%); ¹H NMR (CDCl₃, 300 MHz) δ 9.90 (s, 1H), 7.80 (d, 2H), 7.30 (dd, 4H), 6.90 (d, 2H), 4.20 (t, 2H), 3.10 (t, 2H).

### Preparation of 5-[4-[2-(4-chlorophenyl)ethoxy]phenylmethylene]thiazolidine-2,4-dione (ST1862)

The product was prepared as described in example 1 (method A) from 4-[2-(4-chlorophenyl)ethoxy]benzaldehyde (708 mg, 2.70 mmol) in 20 mL of anhydrous toluene, with thiazolidine-2,4-dione (320 mg, 2.70 mmol), acetic acid (21 mg, 0.35 mmol) and piperidine (29.8 mg, 0.35 mmol), except for the reaction time (5 hours instead of 7 hours). After cooling the mixture, yellow product crystals were separated which were left for 30 minutes at 0° C., then filtered, triturated first with cold toluene and then with water, and then dried. 786 mg of product were obtained (yield=81%); Melting point (Mp)=202-203° C.; TLC: silica gel, eluent CH₂Cl_{2:}CH₃OH 9: 1, Frontal ratio (Fr)=0.6; ¹H NMR (DMSO_{d6}, 300 MHz) δ 7.70 (s, 1H), 7.50 (d, 2H), 7.30 (s, 4H), 7.10 (d, 2H), 4.25 (t, 2H), 3.05 (t, 2H); HPLC: column: LunaC₁₈ (5 μm) (4.6×250 mm), T=30° C., mobile phase: NH₄H₂PO₄ 0.1M:CH₃CN (3:7 v/v), pH=as is, flow rate=1 mL/min, 205 nm UV detector, retention time=11.25 min; Elemental Analysis (E.A.) conforms for C₁₈H₁₄NO₃SCl

### EXAMPLE 21

### Preparation of 5-[4-[2-(4-chlorophenyl)ethoxy]phenylmethyl]-thiazolidine-2,4-dione (ST1864)

To a suspension of ST1862, prepared as described in example 20, (600 mg, 1.67 mmol), in anhydrous MeOH (20 mL), was added piecemeal in small portions Mg in powder form (607 mg, 25.0 mmol). The reaction mixture was left for 5 hours at 25° C. After this time period the solvent was evaporated, water was added to the residue and acidified to pH 2 with a solution of HCl 1 N, and the aqueous phase was extracted with CH₂Cl₂. The pooled organic phases were washed with a saturated solution of NaCl, dried on anhydrous sodium sulphate and evaporated dry in vacuo. The residue thus obtained was purified by silica gel chromatography using CHCl₃:CH₃OH 99.5:0.5 as the eluent to give the still impure product which was recrystallised with methanol to give 180 mg of product (yield=30%); Melting point (Mp)=147-148° C.; TLC: silica gel, eluent CHCl_{3:}CH₃OH 9.95:0.05, Frontal ratio (Fr)=0.16; ¹H NMR (PMSO_{d6}, 300 MHz) δ 12.00 (brs, 1H), 7.40 (s, 4H), 7.20 (d, 2H), 6.90 (d, 2H), 4.90 (m, 1H), 4.20 (t, 2H), 3.30 (m, 2H), 3.00 (m, 2H); HPLC: column: LunaC₁₈ (5 μm) (4.6×250 mm), T=30° C., mobile phase: NH₄H₂PO₄ 0,05M:CH₃CN (4:6 v/v), pH=4, flow rate 1 mL/min, 205 nm UV detector, retention time=14.31 min; Elemental Analysis (E.A.) conforms for C₁₈H₁₆NO₃SCl.

### EXAMPLE 22

### Preparation of dimethyl 3-[2-(4-chlorophenyl)ethoxy]-benzylmalonate (ST1863)

### Preparation of the intermediate product dimethyl 3-hydroxybenzylidenemalonate

The product was prepared as described in example 1 (method A) starting from 3-hydroxybenzaldehyde (3.02 g, 24.7 mmol), dimethylmalonate (2.83 mL, 24.7 mmol), piperidine (314 mg, 3.68 mmol) and glacial acetic acid (221 mg, 3.68 mmol), except for the reaction time (5 hours instead of 7). 3.91 g of product were obtained after purification (yield=67%); ¹H NMR (CDCl₃, 300 MHz) δ 7.80 (s, 1H), 7.30 (m, 1H), 6.90 (m, 3H), 3.90 (s, 6H).

Preparation of the intermediate product dimethyl 3-hydroxybenzylmalonate

3-Hydroxybenzylidenemalonate (1.51 g, 6.40 mmol) was solubilised in 40 mL of methanol and added with 151 mg of 10% Pd/C. The mixture was then subjected to catalytic hydrogenation at 50 psi at ambient temperature for 18 hours. After this time period the mixture was filtered on celite and the organic phase evaporated in vacuo. The residue thus obtained was purified by silica gel chromatography using hexane:ethyl acetate 8:2 as the eluent. 1.31 g of product were obtained (yield=86%); 1H NMR (CDCl₃, 300 MHz) δ7.20 (t, 1H), 6.80 (m, 3H), 3.60 (s, 7H), 3.20 (d, 2H).

### Preparation of dimethyl 3-[2-(4-chlorophenyl)ethoxy]benzyl-malonate (ST1863)

The product was prepared as described in example 14 (method C) starting from 3-hydroxybenzylmalonate (664 mg, 2.80 mmol), 2-(4-chlorophenyl)ethanol (435 mg, 2.80 mmol), triphenylphosphine (953 mg, 3.64 mmol), and DEAD (572 μL, 3.64 mmol) except for the reaction time (one night instead of 5 days). 700 mg of product were obtained after purification (yield=66%); TLC: silica gel, eluent: hexane:ethyl acetate 8:2, Frontal ratio (Fr)=0.35; ¹H NMR (CDCl₃, 300 MHz) δ 7.20 (m, 5H), 6.70 (m, 3H), 4.10 (t, 2H), 3.70 (s, 6H), 3.65 (t, 1H), 3.20 (d, 2H), 3.00 (t, 2H); HPLC: column: Luna C₁₈ (5 μm) (4.6×250 mm), T=30° C., mobile phase: NH₄H₂PO₄ 0,05M:CH₃CN (4:6 v/v), pH=4, flow rate 1 mL/min, 205 nm UV detector, retention time=25.72 min; Elemental Analysis (E.A.) conforms for C₂₀H₂₁ Cl O₅.

### EXAMPLE 23

### Preparation of dimethyl 3-[2-(phenyl)ethoxy]benzlmalonate (ST1895)

ST1863, prepared as described in example 22 (470 mg, 1.20 mmol), was dissolved in 25 mL of methanol and subjected to catalytic hydrogenation at 60 psi with 10% Pd/C (50 mg) for 72 hours at ambient temperature. The suspension was filtered on celite, and the filtrate was evaporated in vacuo to give 95 mg of product (yield=22%); TLC: silica gel, eluent hexane:ethyl acetate 8:2, Frontal ratio (Fr)=0.29; ¹H NMR (CDCl₃, 300 MHz) δ 7.30 (m, 6H), 6.75 (m, 3H), 4.15 (t, 2H), 3.70 (s+t, 7H), 3.20 (d, 2H), 3.10 (t, 2H); HPLC: column: Inertisil ODS-3 (5 μm) (4.6×250 mm), T=30° C., mobile phase CH₃CN:H₂O (70:30 v/v), pH=3.5, flow rate=0.75 mL/min, 205 nm TV detector, retention time=13.63 min; KF=0.4% H₂O; Elemental Analysis (E.A.) conforms for C₂₀H₂₂O₅.

### EXAMPLE 24

### Preparation of dimethyl 3-[N-(4-trifluoromethylbenzyl)carbamoyl]-4-methoxybenzylmalonate (ST 1933)

### Preparation of the intermediate product methyl 5-formyl-2-methoxybenzoate acid

The product was prepared according to the procedure described in EP 0846693A1 starting from 5-formylsalicylic acid (2.00 g, 12.0 mmol) and iodomethane (10.2 g, 72.0 mmol) in DMF (45 mL) with K₂CO₃ (3.50 g, 25.2 mmol) to obtain 1.59 g of product (yield=68%) with analytical data coinciding with those reported in the reference literature.

### Preparation of the intermediate product 5-formyl-2-methoxybenzoic acid

The product was prepared according to the procedure described in EP 0846693A1 starting from methyl 5-formyl-2-methoxybenzoate (2.35 g, 12.1 mmol) in absolute AcOH (33 mL) with concentrated HCl (33 mL) to obtain 1.59 g of product (yield=73%) with analytical data coinciding with those reported in the reference literature.

Preparation of the intermediate product dimethyl-3-carboxy-4-methoxybenzylidenemalonate

The product was prepared according to the procedure described in example 1 (method A) starting from 5-formyl-2-methoxybenzoic acid (800 mg, 4.44 mmol) in 32 mL of anhydrous toluene, with dimethylmalonate (586 mg, 4.44 mmol), piperidine (57 mg, 0.67 mmol) and glacial acetic acid (40.2 mg, 0.67 mmol), except for the reaction time (5 hours instead of 7). At the end of this time period the mixture was cooled and, after 30 minutes at 4° C., crystals were separated which were filtered and triturated several times with toluene. 870 mg of product were obtained (yield=67%); ¹H NMR (DMSO_{d6}, 300 MHz) δ 7.90 (s, 1H), 7.80 (s, 1H), 7.70 (d, 1H), 7.20 (d, 1H), 3.90 (s, 3H), 3.80 (d, 6H).

### Preparation of the intermediate product dimethyl 3-[N-(4-trifluoromethylbenzyl) carbamoyl]4-methoxybenzylidenemalonate

Method E

To the solution of dimethyl-3-carboxy-4-methoxybenzylidenemalonate (620 mg, 2.10 mmol) in anhydrous DMF (6.2 mL) were added under N₂ flow 4-trifluoromethylbenzylamine (368 mg, 2.10 mmol), diethylphosphorocyanidate (377 mg, 2.10 mmol) and triethylamine (234 mg, 2.31 mmol). The reaction mixture was left at ambient temperature under N₂ flow for 24 hours. After this time period the reaction mixture was poured into water and extracted with ethyl acetate. The organic phase was then washed with HCl 1N, NaOH 1N and water, dried on anhydrous sodium sulphate and evaporated in vacuo. The residue thus obtained was purified by silica gel chromatography using hexane:ethyl acetate 6:4 as the eluent. 249 mg of product were obtained (yield=26%); 1H NMR (CDCl₃, 300 MHz) δ8.30 (s, 1H), 8.10 (brs, 1H), 7.70 (s, 1H), 7.50 (m, 5H), 6.90 (d, 1H), 4.70 (d, 2H), 3.90 (s, 3H), 3.80 (d, 6H).

### Preparation of dimethyl 3-[N-(4-trifluoromethylbenzyl)-carbamoyl]4-methoxybenzylmalonate (ST1933)

Dimethyl 3-[N-(4-trifluoromethylbenzyl)carbamoyl]4-methoxybenzylidenemalonate (148 mg, 0.33 mmol) was solubilised in methanol (18 mL) and added with 74 mg of 10% Pd/C. The mixture thus obtained was hydrogenated at 57 psi for 18 hours at ambient temperature. After this time period the suspension was filtered on celite and the filtrate dried by evaporating the solvent in vacuo to give 140 mg of product as a white solid (yield=94%); Melting point (Mp)=126-128° C.; TLC: silica gel, eluent hexane:ethyl acetate 6:4, Frontal ratio (Fr)=0.2; ¹H NMR (CDCl₃, 300 MHz) δ 8.30 (m, 1H), 8.10 (d, 1H), 7.60 (d, 2H), 7.50 (d, 2H), 7.30 (dd, 1H), 6.90 (d, 1H), 4.70 (d, 2H), 3.90 (s, 3H), 3.70 (s+t, 7H), 3.20 (d, 2H). HPLC: column: Inertisil-ODS 3 (5 μm) (4.6×250 mm), T=30° C., mobile phase CH₃CN:H₂O (70:30 v/v), flow rate=0.75 mL/min, 205 nm UV detector, retention time=8.85 min; KF=1.55% H₂O; Elemental Analysis (E.A.) conforms for C₂₂H₂₂F₃NO₆.

### EXAMPLE 25

### Preparation of dimethyl 4-methoxy-3-[2-(4-chlorophenyl)-ethoxy]benzylmalonate (ST1861)

### Preparation of the intermediate product dimethyl 3-hydroxy-4-methoxybenzylidenemalonate

The product was prepared according to the procedure described in example 1 (method A) starting from 3-hydroxy-4-methoxybenzaldehyde (3.00 g, 19.7 mmol), dimethylmalonate (2.60 g, 19.7 mmol), piperidine (251 mg, 2.95 mmol) and glacial acetic acid (177 mg, 2.95 mmol) in 120 mL of anhydrous toluene, except for the eluent used in the purification by chromatography (hexane:ethyl acetate 8:2 instead of 7:3). 5.20 g of product were obtained (yield=98%); ¹H NMR (CDCl₃, 300 MHz) δ 7.70 (s, 1H), 7.00 (m, 2H), 6.90 (d, 1H), 5.60 (brs, 1H), 4.00 (s, 3H), 3.90 (s, 3H), 3.80 (s, 3H).

Preparation of the intermediate product dimethyl 3-hydroxy-4-methoxybenzylmalonate

Dimethyl 3-hydroxy-4-methoxybenzylidenemalonate (5.20 g, 19.5 mmol) in 180 mL of methanol was hydrogenated at 60 psi with 10% Pd/C (520 mg) for 18 hours at ambient temperature. After this time period the reaction mixture was filtered on celite and the solvent was evaporated in vacuo. 4.90 g of product were obtained (yield=93.5%); ¹H NMR (CDCl₃, 300 MHz) δ 6.70 (m, 3H), 3.90 (s, 3H), 3.70 (s, 6H), 3.60 (t, 1H), 3.20 (d, 2H).

### Preparation of dimethyl 4-methoxy-3-[2-(4-chlorophenyl)-ethoxy]benzlmalonate (ST1861)

The product was prepared according to the procedure described in example 14 (method C) starting from dimethyl 3-hydroxy-4-methoxybenzylmalonate (900 mg, 3.38 mmol) with 2-(4-chlorophenyl)ethanol (582 mg, 3.79 mmol), triphenylphosphine (1.15 g, 4.39 mmol) and DEAD (765 mg, 4.39 mmol) in 9 mL of anhydrous THF, except for the reaction time (one night instead of 5 days) and the eluent used in the purification by chromatography (hexane:ethyl acetate 7:3 instead of 8:2). 550 mg of product were obtained (yield=40%); Melting point (Mp)=55-56° C.; TLC: silica gel, eluent hexane:ethyl acetate 7:3, Frontal ratio (Fr)=0.8; ¹H NMR (CDCl₃, 300 MHz) δ 7.25 (m, 4H), 6.75 (m, 3H), 4.20 (t, 2H), 3.80 (s, 3H), 3.70 (s, 6H), 3.60 (t, 1H), 3.10.(m, 4H); HPLC: column: Symmetry C₁₈ (5 μm) (3.9×150 mm), T=30° C., mobile phase CH₃CN:NH₄H₂PO₄ (50:50 v/v), flow rate 0.75 mL/min, pH=3.2, 205 nm UV detector, retention time=23.23 min; Elemental Analysis (E.A.) conforms for C₂₁H₂₃ClO₆.

### EXAMPLE 26

### Preparation of dimethyl 3-(2-phenylethoxy)-4-methoxy benzyl-malonate (ST1892)

To a solution of ST1861 (475 mg, 1.16 mmol), prepared as described in example 25, in 25 mL of methanol, was added 10% Pd/C (48 mg) and the resulting suspension was left under H₂ at 50 psi for 2 days at ambient temperature. After this time period the suspension was filtered on celite and the solvent evaporated in vacuo. The residue obtained was purified by silica gel chromatography using hexane:ethyl acetato 8:2 as the eluent to give 130 mg of product yield=30%); TLC: silica gel, eluent hexane:ethyl acetate 6:4, Frontal ratio (Fr)=0.55; ¹H NMR (CDCl₃, 300 MHz) δ 7.30 (m, 5H), 6.75 (m, 3H), 4.20 (t, 2H), 3.80 (s, 3H), 3.70 (s, 6H), 3.60 (t, 1H), 3.10 (m, 4H); HPLC: column: Inertisil ODS-3 (5 μm) (4.6×250 mm), T=30° C., mobile phase CH₃CN:NH₄H₂PO₄ 50 mM (50:50 v/v), flow rate=0.75 mL/min, pH=3.2, 205 nm UV detector, retention time=8.92 min; Elemental Analysis (E.A.) conforms for C₂₁H₂₄O₆.

### EXAMPLE 27

### Preparation of dimethyl 4-[2-(4-methoxyphenyl)ethoxy]benzyl-malonate (ST1893)

The product was prepared as described in example 14 (method C) starting from dimethyl 4-hydroxybenzylmalonate, prepared as described in example 13 (600 mg, 2.52 mmol), 2-(4-methoxyphenyl)-ethanol (383 mg, 2.52 mmol), DEAD (568 mg, 3.27 mmol) and triphenylphosphine (856 mg, 3.27 mmol) in 15 mL of THF, except for the reaction time (one night instead of 5 days). 277 mg of product were obtained (yield=29.5%); TLC: silica gel, eluent hexane:ethyl acetate 8:2; Frontal ratio (Fr)=0.2; ¹H NMR (CDCl₃, 300 MHz) δ 7.20 (d, 2H), 7.10 (d, 2H), 6.80 (m, 4H), 4.10 (t, 2H), 3.80 (s, 3H), 3.70 (s, 6H), 3.60 (t, 1H), 3.15 (d, 2H), 3.00 (t, 2H); HPLC: Column: Inertisil ODS-3 (5 μm) (4.6×250 mm), T=30° C., mobile phase CH₃CN:H₂O (60:40 v/v), flow rate 0.75 mL/min, pH=as is, 205 nm UV detector, retention time=23.93 min; Elemental Analysis (E.A.) conforms for C₂₁H₂₄O₆.

### EXAMPLE 28

### Preparation of dimethyl 4-[3-(4-methoxyphenyl)propyloxy]-benzylmalonate (ST1894)

The product was prepared as described in example 14 (method C) starting from dimethyl 4-hydroxybenzylmalonate (600 mg, 2.52 mmol), prepared as described in example 13, with 3-(4-methoxyphenyl)-1-propanol (419 mg, 2.52 mmol), DEAD (568 mg, 3.27 mmol) and triphenylphosphine (857 mg, 3.27 mmol), in 15 mL of anhydrous THF, except for the reaction time which was one night instead of 5 days. 400 mg of product were obtained (yield=41.1%); TLC: silica gel, eluent hexane:ethyl acetate 8:2; Frontal ratio (Fr)=0.22; ¹H NMR (CDCl₃, 300 MHz) δ 7.10 (dd, 4H), 6.80 (dd, 4H), 3.90 (t, 2H), 3.80 (s,3H), 3.70 (s, 6H), 3.60 (t, 1H), 3.20 (d, 2H), 2.70 (t, 2H), 2.00 (m, 2H); HPLC: column: Inertisil ODS-3 (5 μm) (4.6×250 mm), T=30° C., mobile phase CH₃CN:H₂O (60:40 v/v), flow rate 0.75 mL/min, pH=as is, 205 nm UV detector, retention time=32.46 min; KF=0.15% H₂O; Elemental Analysis (E.A.) conforms for C₂₂H₂₆O₆.

### EXAMPLE 29

### Preparation of dimethyl 4-[2-(2-naphthyl)ethoxy]benzyl-malonate (ST1985)

The product was prepared according to the procedure described in example 14 (method C) starting from dimethyl 4-hydroxybenzylmalonate (476 mg, 2 mmol), prepared as described in example 13, 2-naphthalene-ethanol (344 mg, 2 mmol), DEAD (451 mg, 2,6 mmol) and triphenylphosphine (681 mg, 2,6 mmol), in 15 mL of anhydrous THF, except for the reaction time which was 2 days instead of 5 days and the eluent used in the purification by chromatography (hexane:ethyl acetate 9:1 instead of 8:2). The product thus obtained was further purified by crystalisation with isopropanol. 167 mg of product were obtained (yield=21.3%); Melting point (Mp)=68.5° C.; TLC: silica gel, eluent hexane:ethyl acetate 8:2; Frontal ratio (Fr)=0.7; ¹H NMR (CDCl₃, 300 MHz) δ 7.80 (m, 4H), 7.40 (m, 3H), 7.10 (d, 2H), 6.90 (d, 2H), 4.20 (t, 2H), 3.70 (s, 6H), 3.60 (t, 1H), 3.20 (t, 2H), 3.10 (d, 2H); HPLC: Column: Symmetry-C₁₈ (3.5 μm) (4.6×75 mm), T=ambient, mobile phase CH₃CN:H₂O (60:40 v/v), flow rate 0.9 mL/min, pH=as is, 205 nm UV detector, retention time=10.80 min; KF=0.3% H₂O; Elemental Analysis (A.E.) conforms for C₂₄H₂₄O₅.

### EXAMPLE 30

### Preparation of ethyl (2S)-2-benzoylamino-3-[4-[(4-methoxybenzyl)carbamoyl]oxyphenyl]propanoate (ST 1500)

Method D

The product was prepared from 4-methoxy benzylisocyanate (400 mg, 2.24 mmol) and N-benzoyl-L-tyrosine ethyl ester (700 mg, 2.24 mmol) dissolved in anhydrous THF (5 mL). NEt₃ (20 μL) was added to the solution and the reaction was left to stir for 18 hours at ambient temperature. The solution was evaporated to give 980 mg of product as a white solid (yield=92%); Melting point (Mp)=149-151° C.; [a]_{D}²⁰=+69.3 (c=0.5% in CHCl₃); TLC: silica gel, eluent AcOEt:CH₂Cl₂ 2:8, Frontal ratio (Fr)=0.61; ¹H NMR (CDCl₃, 300 MHz) δ 7.80 (d, 2H), 7.50 (m, 3H), 7.30 (d, 2H), 7.10 (dd, 4H), 6.90 (d, 2H), 6.60 (d, 1H), 5.30 (m, 1H), 5.05 (q, 1H), 4.40 (d, 2H), 4.20 (q, 2H), 3.80 (s, 3H) 3.25 (m, 2H), 1.30 (t, 3H); HPLC: column: Symmetry (5 μm) (250×4.6 mm), mobile phase CH₃CN:KH₂PO₄ 50 mM (50:50 v/v), pH=as is, T=30° C., flow rate=0.75 mL/min, 205 nm UV detector, retention time=19.16 min; KF=0.8% H₂O; Elemental Analysis (E.A.) conforms for C₂₇H₂₈N₂O₆.

### EXAMPLE 31

### Preparation of dimethyl 4-[[(4-methoxybenzyl) carbamoyl]oxy]-benzylmalonate (ST1538)

The product was prepared as described in example 30 (method D) starting from 4-methoxy benzylisocyanate (400 mg, 2.58 mmol) and dimethyl 4-hydroxybenzylmalonate, prepared as described in example 13 (700 mg, 3.02 mmol) in anhydrous THF (10 mL) and NEt₃ (20 μL), except for the fact that the residue obtained after evaporation of the reaction solvent was purified by flash chromatography on silica gel, using AcOEt:hexane 3:7 as the eluent, to give 740 mg of white solid (yield=72%); Melting point (Mp)=78.6° C.; TLC: silica gel, eluent AcOEt:hexane 3:7, Frontal ratio (Fr)=0.22; ¹H NMR (CDCl₃, 300 MHz) δ 7.22 (d, 2H), 7.20 (d, 2H), 7.10 (d, 2H), 6.90 (d, 2H), 5.20 (m, 1H), 4.40 (d, 2H), 3.80 (s, 3H) 3.70 (s, 6H), 3.60 (t, 1H), 3.20 (d, 2H); HPLC: column: Symmetry (5 μm)-(250×4.6 mm), mobile phase CH₃CN:H₂O (50:50 v/v), pH=as is, T=30° C., flow rate=0.75 mL/min, 205 nm UV detector, retention time=16.12 min; Elemental Analysis (E.A.) conforms for C₂₁H₂₃NO₇.

### EXAMPLE 32

### Preparation of dimethyl 4-[[(4-trifluorotolyl)carbamoyl]oxy]benzylmalonate (ST1620)

The product was prepared as described in example 30 (method D) starting from 4-trifluorotolyl isocyanate (410 mg, 2.19 mmol) and dimethyl 4-hydroxybenzylmalonate, prepared as described in example 13 (600 mg, 2.52 mmol) in anhydrous THF (10 mL) and NEt₃ (20 μL), except for the fact that the residue obtained after evaporation of the reaction solvent was purified by flash chromatography on silica gel, using AcOEt:hexane 3:7 as the eluent, to give 350 mg of product as a white solid (yield=37.1%); Melting point (Mp)=109.1° C.; TLC: silica gel, eluent AcOEt:hexane 3:7, Frontal ratio (Fr)=0.44; ¹H NMR (CDCl₃, 300 MHz) δ 7.60 (q, 4H), 7.20 (d, 2H), 7.10 (d, 3H), 3.70 (s, 6H), 3.60 (t, 1H), 3.20 (d, 2H); HPLC: column: Symmetry (5 μm) (250×4.6 mm), mobile phase CH₃CN:H₂O (60:40 v/v), pH=as is, T=30° C., flow rate=0.75 mL/min, 205 nm UV detector, retention time=16.44 min; Elemental Analysis (E.A.) conforms for C₂₀H₁₈F₃NO₆.

### EXAMPLE 33

### Preparation of dimethyl 4-[[(2,4-dichlorophenyl)carbamoyl]oxy]benzylmalonate (ST1818)

The product was prepared as described in example 30 (method D) starting from 2,4-dichlorophenylisocyanate (73 mg, 0.38 mmol) and dimethyl 4-hydroxybenzylmalonate, prepared as described in example 13 (100 mg, 0.42 mmol) in anhydrous THF (3 mL), with NEt₃ (10 μL), except for the fact that the residue obtained after evaporation of the reaction solvent was purified by flash chromatography on silica gel, using AcOEt:hexane 2:8 as the eluent, to give 120 g of product as a white solid (yield=74%); Melting point (Mp)=84° C.; TLC: silica gel, eluent AcOEt:hexane 3:7, Frontal ratio (Fr)=0.39; ¹H NMR (CDCl₃, 300 MHz) δ 8.10 (brd, 1H), 7.40 (m, 2H), 7.22 (m, 3H), 7.15 (d, 2H), 3.70 (s+t, 7H), 3.20 (d, 2H); HPLC: column: Inertisil ODS-3 (5 μm)-(250×4.6 mm), mobile phase CH₃CN:H₂O (60:40 v/v), pH=as is, T=30° C., flow rate=0.75 mL/min, 205 nm UV detector, retention time=28.13 min; Elemental Analysis (E.A.) conforms for C₁₉H₁₇Cl₂NO₆.

### EXAMPLE 34

### Preparation of dimethyl 4-[[(4-chlorophenyl)carbamoyl]oxy]benzylmalonate (ST1696)

The product was prepared as described in example 30 (method D) starting from 4-chlorophenylisocyanate (560 mg, 3.65 mmol) and dimethyl 4-hydroxybenzylmalonate, prepared as described in example 13, (1.00 g, 4.20 mmol) in anhydrous THF (16.6 mL), with NEt₃ (20 μL), except for the fact that after evaporation of the solvent the reaction residue was dissolved in AcOEt (130 mL) and extracted with a solution of NaOH 0.1 N (3×50 mL). The residue obtained after evaporation of the solvent was purified by flash chormatography on silica gel, using AcOEt:hexane 2:8 as the eluent to give 550 mg of product as a white solid (yield=38%); Melting point (Mp)=125-127° C.; TLC: silica gel, eluent AcOEt:hexane 3:7, Frontal ratio (Fr)=0.37; ¹H NMR (CDCl₃, 300 MHz) δ 7.40 (d+s, 2H), 7.30-7.20 (m, 4H), 7.10 (d, 2H), 6.90 (brs, 1H), 3.70 (s, 6H), 3.65 (t, 1H), 3.20 (d, 2H); HPLC: column: Symmetry C₁₈ (5 μm )-(250×4.6 mm), mobile phase CH₃CN:H₂O (65:35 v/v), pH=as is, T=30° C., flow rate=0.75 mL/min, 205 nm UV detector, retention time=14.78 min; Elemental Analysis (E.A.) conforms for C₁₉H₁₈ClNO₆.

### EXAMPLE 35

### Preparation of dimethyl 4-[2-(pyridinio)ethoxy]benzyl-malonate methanesulphonate (ST1799)

### Preparation of the intermediate product dimethyl 4-[2-(hydroxy)ethoxy]benzylidenemalonate

To dimethyl 4-hydroxybenzylidenemalonate (2.00 g, 8.47 mmol) in anhydrous DMF (40 mL) was added NaH (244 mg, 10.2 mmol) and after approximately 30 minutes 2-bromoethanol (1.37 g, 11.0 mmol). The reaction mixture was left at a temperature of 70° C. for 24 hours. After this time period H₂O (200 mL) was added to the mixture and the aqueous phase was extracted with ethyl acetate (2×100 mL). The organic phase washed with H₂O (2×50 mL) was dried on anhydrous Na₂SO₄ and then evaporated to give 2.00 g of oily product (yield=84%); ¹H NMR (CDCl₃, 300 MHz) δ 7.70 (s, 1H), 7.40 (d, 2H), 6.90 (d, 2H), 4.10 (t, 2H), 4.00 (t, 2H), 3.85 (d, 6H).

### Preparation of the intermediate product dimethyl 4-[2-(hydroxy)ethoxy]benzylmalonate

The product was prepared from dimethyl 4-[2-(hydroxy)ethoxy]benzylidenemalonate (4.50 g, 16.0 mmol) by catalytic hydrogenation with 10% Pd/C (500 mg) in MeOH (120 mL) in an H₂ atmosphere (50 psi) for 24 hours. After this time period, the solution was filtered on celite and the solvent evaporated to give 4.20 g of oily product (yield=93%); ¹H NMR (CDCl₃, 300 MHz) δ 7.10 (d, 2H), 6.85 (d, 2H), 4.10 (t, 2H), 3.95 (t, 2H), 3.70 (s, 3H), 3.65 (t, 1H), 3.20 (d, 2H).

### Preparation of the intermediate product dimethyl 4-[2-(methanesulphonyl)ethoxy]benzylmalonate

To dimethyl 4-[2-(hydroxy)ethoxy]benzylmalonate (2.00 g, 7.00 mmol) in CH₂Cl₂ (50 mL) were added anhydrous pyridine (1.66 g, 21.0 mmol) and mesyl chloride (2.43 g, 21.0 mmol), dropwise at 0° C. At the end of the additions the mixture was left at 50° C. for 6 hours. After evaporation of the solvent the residue was re-dissolved in AcOEt (100 mL) and the organic phase was washed with H₂O (2×50 mL), then with HCl 1N (2×50 mL) and again with H₂O to neutral pH. The organic phase dried on anhydrous Na₂SO₄ was evaporated to give 2.02 g of oily product (yield=80%); ¹H NMR (CDCl₃, 300 MHz) δ 7.10 (d, 2H), 6.85 (d, 2H), 4.60 (t, 2H), 4.22 (d, 2H), 3.70 (s, 3H), 3.65 (t, 1H), 3.20 (d, 2H), 3.10 (s, 3H).

### Preparation of dimethyl 4-[2-(pyridinio)ethoxy]benzylmalonate methanesulphonate (ST1799)

Method F

The product was prepared from dimethyl 4-[2-(methanesulphonyl)ethoxy]benzylmalonate (960 mg, 2.60 mmol) dissolved in pyridine (15 mL). The reaction mixture was left for 18 hours at 75° C. After evaporation of the solvent the oily residue was washed with diethyl ether. The still impure final residue was purified by flash chromatography on silica gel using CHCl₃:MeOH 5:5 as the eluent to give 940 mg of oily product (yield=82.3%); TLC: silica gel, eluent CHCl 4.2:CH₃OH 2.8:isopropanol 0.7:CH₃COOH 1.05:H₂O 1.05, Frontal ratio (Fr)=0.48; ¹H NMR (CDCl₃, 300 MHz) δ 9.40 (brd, 2H), 8,42 (brt, 1H), 8.00 (brd, 2H), 7.05 (d, 2H), 6.75 (d, 2H), 5.35 (m, 2H), 4.5 (m, 2H), 3.70 (s, 6H), 3.60 (t, 1H), 3.10 (d, 2H), 2.80 (s, 3H); HPLC: column: Spherisorb-SCX (5 μm) (250×4.6 mm), mobile phase CH₃CN:NH₄H₂PO₄ 50 mM (40:60 v/v), pH=3.5, T=30° C., flow rate=0.75 mL/min, 205 nm UV detector, retention time=18.65 min; KF=4.5% H₂O; Elemental Analysis (E.A.) conforms for C₁₉H₂₂NO₅.CH₃O₃S.

### EXAMPLE 36

### Preparation of dimethyl 4-[[(4-nitrophenyl)carbamoyl]oxy]-benzylmalonate (ST1865)

The product was prepared as described in example 30 (method D) starting from dimethyl 4-hydroxybenzylmalonate, prepared as described in example 13 (180 mg, 0.75 mmol), 4-nitrophenylisocyanate (124 mg, 0.75 mmol) in anhydrous THF (4 mL) and NEt₃ (20 μL), except for the fact that the residue obtained after evaporation of the reaction solvent was purified by flash chromatography on silica gel using hexane:AcOEt 1:1 as the eluent. 221 mg of product were obtained (yield=73%); Melting point (Mp)=128-130° C.; TLC: silica gel, eluent hexane:AcOEt 1:1, Frontal ratio (Fr)=0.55; ¹H NMR (CDCl₃, 300 MHz) δ 8.20 (d, 2H), 7.60 (d, 2H), 7.30 (d, 2H), 7.10 (d, 2H), 3.70 (s+t, 7H), 3.25 (d, 2H); HPLC: column: luna C₁₈, (5 μm) (4.6×250 mm), T=30° C., mobile phase NH₄H₂PO₄ 0,05M:CH₃CN 4:6 (v/v), pH=4, flow rate=1 mL/min, 205 nm UV detector, retention time=8.56 min; Elemental Analysis (E.A.) conforms for C₁₉H₁₈N₂O₈.

### EXAMPLE 37

### Preparation of dimethyl 3-[[(4-methoxybenzyl)carbamoyl]oxy]benzylmalonate (ST1907)

The product was prepared as described in example 30 (method D) starting from dimethyl 3-hydroxybenzylmalonate, prepared as described in example 22 (200 mg, 0.84 mmol), p-methoxybenzylisocyanate (188 mg, 1.16 mmol) and NEt₃ (20 μL) in anhydrous THF (5 mL), except for the reaction time which was 72 hours instead of 18 hours and for the fact that after evaporation of the solvent in vacuo the residue was purified by silica gel chromatography using hexane:AcOEt 7:3 as the eluent. 181 mg of product were obtained (yield=54%); Melting point (Mp)=62-64° C.; TLC: silica gel, eluent hexane:AcOEt 6:4, Frontal ratio (Fr)=0.36; ¹H NMR (CDCl₃, 300 MHz) δ 7.30 (m, 4H), 7.00 (m, 2H), 6.90 (d, 2H), 5.20 (brm, 1H), 4.40 (m, 2H), 3.80 (s, 3H), 3.70 (s+t, 7H), 3.20 (d, 2H); HPLC: column: Symmetry-C₁₈, (5 μm) (4.6×250 mm), T=30° C., mobile phase CH₃CN:H₂O 1:1 (v/v), pH=as is, flow rate=0.75 mL/min, 205 nm UV detector, retention time=17.58 min; KF=0.18% H₂O; Elemental Analysis (E.A.) conforms for C₂₁H₂₃NO₇.

### EXAMPLE 38

### Preparation of dimethyl 3-[[(4-butylphenyl)carbamoyl]oxy]-benzlmalonate (ST1908)

The product was prepared as described in example 30 (method D) starting from dimethyl 3-hydroxybenzylmalonate, prepared as described in example 22 (200 mg, 0.84 mmol), p-butylphenylisocyanate (174 mg, 1.0 mmol) and 20 μL of NEt₃ in 5 mL of anhydrous THF, except for the fact that after 36 hours a further 52.5 mg (0.30 mmol) of p-butylphenylisocyanate were added and the reaction was left at ambient temperature for another 4 days. The solvent was evaporated in vacuo and the residue purified by silica gel chromatography using hexane:AcOEt 8:2 as the eluent. 130 mg of product were obtained Wield=37.5%); Melting point (Mp)=53-54° C.; TLC: silica gel, eluent hexane:AcOEt 8:2, Frontal ratio=0.26; ¹H NMR (CDCl₃, 300 MHz) δ 7.30 (d, 1H), 7.20 (m, 2H), 7.10 (m, 5H), 6.80 (brs, 1H), 3.70 (s, 6H) 3.65 (t, 1H), 3.20 (d, 2H) 2.60 (t, 2H), 1.60 (m, 2H), 1.30 (m, 2H), 0.90 (t, 3H); HPLC: column: Symmetry-C₁₈, (5 μm) (4.6×250 mm), T=30° C., mobile phase CH₃CN:H₂O 7:3 (v/v), pH=as is, flow rate=0.75 mL/min, 205 nm UV detector, retention time=16.17 min; Elemental Analysis (E.A.) conforms for C₂₃H₂₇NO₆.

### EXAMPLE 39

### Preparation of dimethyl 4-[[(4-butylphenyl)carbamoyl]oxy]-benzylmalonate (ST1909)

The product was prepared as described in example 30 (method D) starting from dimethyl 4-hydroxybenzylmalonate, prepared as described in example 13 (200 mg, 0.84 mmol), p-butylphenylisocyanate (220 mg, 1.26 mmol) and NEt₃ (20 μL) in 5 mL of anhydrous THF, except for the reaction time which was 24 hours instead of 18 hours and the fact that after evaporation of the solvent in vacuo the product was purified by silica gel chromatography using hexane:AcOEt 8:2 as the eluent to give 129 mg of product (yield=37%); Melting point (Mp)=90-92° C.; TLC: silica gel, eluent hexane:AcOEt 8:2, Frontal ratio (Fr)=0.23; ¹H NMR (CDCl₃, 300 MHz) δ 7.30 (m, 3H), 7.10 (d, 2H), 7.00 (m, 3H), 6.80 (brs, 1H), 3.70 (s, 6H) 3.65 (t, 1H), 3.25 (d, 2H), 2.60 (t, 2H), 1.60 (m, 2H), 1.35 (m, 2H), 0.90 (t, 3H); HPLC: column: Symmetry-C₁₈, (5 μm) (4.6×250 mm), T=30° C., mobile phase CH₃CN:H₂O 7:3 (v/v), pH=as is, flow rate=0.75 mL/min, 205 nm UV detector, retention time=15.96 min; KF=0.52% H₂O; Elemental Analysis (E.A.) conforms for C₂₃H₂₇NO6.

### EXAMPLE 40

### Preparation of dimethyl 3-[[(4-chlorophenyl)carbamoyl]oxy]-benzylmalonate (ST1856)

The product was prepared as described in example 30 (method D) starting from dimethyl 3-hydroxybenzylmalonate (800 mg, 3.36 mmol) prepared as described in example 22, 4-chlorophenylisocyanate (774 mg, 5.04 mmol) and NEt₃ (20 μL) in 30 mL of anhydrous THF, except for the fact that after evaporating the solvent in vacuo, the residue was treated with ethyl acetate, filtered and the filtrate evaporated in vacuo. The residue obtained was purified by two silica gel chromatographies, the first using CHCl₃:hexane 8:2 and the second hexane:ethyl acetate 7:3 as the eluent to give 520 mg of product (yield=39.6%); Melting point (Mp)=79-80° C.; TLC: silica gel, eluent hexane:ethyl acetate 6:4, Frontal ratio (Fr)=0.6; ¹H NMR (CDCl₃, 300 MHz) δ 7.40 (d, 1H), 7.30 (m, 3H), 7.10 (m, 2H), 6.90 (brs, 1H), 3.70 (s+t, 7H), 3.25 (d, 2H); HPLC: column: Luna C₁₈ (5 μm) (4.6×75 mm), T=50° C., mobile phase NaH₂PO₄ 0,05M:CH₃CN (50:50 v/v), flow rate=1 mL/min, pH=as is, 205 nm UV detector, retention time=24.34 min; Elemental Analysis (E.A.) conforms for C₁₉H₁₈ClNO₆.

### EXAMPLE 41

### Preparation of (Z)-2-ethoxy-3-[4-[2-(4-chlorophenyl)ethoxy]-phenyl]ethyl propenoate (ST2135) and of (E)-2-ethoxy-3-[4-[2-(4-chlorophenyl)ethoxy]phenyl]ethyl propenoate (ST2136)

### Preparation of triethyl phosphonodiazoacetate

The product was prepared as described in Tetrahedron, 1992, 48 (19), 3991-4004 starting from triethyl phosphonoacetate (8.60 g, 38.1 mmol), 80% NaH (1.04 g, 41.86 mmol) and tosylazide (7.50 g, 38.1 mmol) to give 6.60 g of product (yield=69%). The analytical data were as reported in the literature.

### Preparation of triethyl 2-ethoxyphosphonoacetate

The product was prepared according to the procedure described in Tetrahedron, 1992, 48 (19), 3991-4004 starting from triethyl phosphonodiazoacetate (5.00 g, 19.9 mmol), absolute ethanol (36 mL), and bivalent rhodium acetate dimer (88.3 mg, 0.199 mmol) to obtain 3.20 g of product (yield=60%); ¹H NMR (CDCl₃, 300 MHz) δ 4.30-4.20 (m, 7H), 3.70 (dq, 2H), 1.40 (m, 12H).

### Preparation of (Z)-2-ethoxy-3-[4-[2-(4-chlorophenyl)-ethoxy]phenyl]ethyl propenoate (ST2135) and of (E)-2-ethoxy-3-[4-[2-(4-chlorophenyl)ethoxy]phenyl]ethyl propenoate (ST2136)

Method H

Triethyl 2-ethoxyphosphonoacetate (3.1 g, 11.5 mmol) was added at 0° C. to a suspension of 80% NaH (384 mg, 12.78 mmol) in anhydrous THF (20 mL) and after approximately 30 minutes at ambient temperature 4-[2-(4-chlorophenyl)ethoxy]benzaldehyde (2.4 g, 9.2 mmol) was added, prepared as described in example 20, dissolved in anhydrous THF (20 mL). At the end of the addition the reaction mixture was left to stir at ambient temperature for 20 hours. After evaporation of the solvent in vacuo the residue was purified by two SiO₂ gel chromatographies, the first using AcOEt:hexane 2:8, and the second AcOEt:hexane 5:95 as the eluent. 2.70 g of a mixture of the two isomers were obtained (yield=63%), which in subsequent preparations was used as is in the synthesis of ST2211 (example 43) and ST2130 (example 42). To isolate the Z and E isomers, the mixture was further purified by two SiO₂ gel, chromatographies, the first using AcOEt:hexane 5:95 and the second CH₂Cl₂ as the eluent to give 330 mg of ST 2135 (Z isomer) as a semisolid (yield=9.6%) and 380 mg of ST 2136 (E isomer) as an oily product (yield=11%).

Analytical data for ST2135 (Z isomer)

TLC: silica gel, eluent AcOEt:hexane 2:8, Frontal ratio (Fr)=0.32; ¹H NMR (CDCl₃, 300 MHz) δ 7.65 (d, 2H), 7.22 (dd, 4H), 6.95 (s, 1H), 6.85 (d, 2H), 4.30 (q, 2H), 4.20 (t, 2H), 4.00 (q, 2H), 3.10 (t, 2H), 1.40 (t, 6H); HPLC: column: Inertisil ODS-3 C18 (5 μm) (250×4.6 mm), mobile phase CH₃CN:H₂O (85:15 v/v), pH=as is, T=ambient, flow rate=0.9 mL/min, 205 nm UV detector, retention time=16.67 min; Elemental Analysis (E.A.) conforms for C₂₁H₂₃ClO₄.

Analytical Data for ST2136 (E Isomer)

TLC: silica gel, eluent AcOEt:hexane 2:8, Frontal ratio (Fr)=0.36; ¹H NMR (CDCl₃, 300 MHz) δ 7.25 (dd, 4H), 7.10 (d, 2H), 6.80 (d, 2H), 6.10 (s, 1H), 4.20 (q+t, 4H), 3.90 (q, 2H), 3.05 (t, 2H), 1.40 (t, 3H), 1.18 (t, 3H); HPLC: column: Inertisil ODS-3 C18 (5 μm) (250×4.6 mm), mobile phase CH₃CN:H₂O (85:15 v/v), pH=as is, T=ambient, flow rate=0.9 ml/min, 205 nm UV detector, retention time=10.79 min; Elemental Analysis (E.A.) conforms for C₂₁H₂₃ClO₄.

### EXAMPLE 42

### Preparation of (R,S)-2-ethoxy-3-[4-[2-(phenyl)ethoxy]phenyl]ethyl propanoate (ST 2130)

To a solution of a mixture of ST 2135 and ST 2136 (600 mg, 1.6 mmol), obtained as described in example 41, in absolute ethanol (20 mL) was added 10% Pd/C (60 mg) and the mixture was left in an H₂ atmosphere at 40 psi, at ambient temperature for 6 hours. After filtration on celite the solvent was evaporated in vacuo and the residue purified by chromatography on SiO₂ gel using hexane:AcOEt 95:5 as the eluent to give 470 mg of product (yield=86%); TLC: silica gel, eluent AcOEt:hexane 2:8, Frontal ratio (Fr)=0.46; ¹H NMR (CDCl₃, 300 MHz) δ 7.25 (dd, 4H), 7.18 (d, 2H), 6.80 (d, 2H), 4.20 (t, 4H), 3.95 (t, 1H), 3.60 (m, 1H), 3.35 (m, 1H), 3.10 (t, 2H), 2.90 (d, 2H), 1.22 (t, 3H), 1.18 (t, 3H); HPLC: column: Inertisil ODS-3 C18 (5 μm) (250×4.6 mm), mobile phase CH₃CN:H₂O (85:15 v/v), pH=as is, T=ambient, flow rate=0.9 mL/min, 205 nm UV detector, retention time=8.98 min; Elemental Analysis (E.A.) conforms for C₂₁H₂₆O₄.

### EXAMPLE 43

### Preparation of (R,S)-2-ethoxy-3-[4-[2-(4-chlorophenyl)ethoxy]phenylmethyl propanoate (ST 2211)

To a solution of a mixture of ST 2135 and ST 2136 (1.15 g, 3.06 mmol), obtained as described in example 41, in anhydrous methanol (73 mL) were added Mg in powder form (1.17 g) and a few crystals of I₂, and the mixture was left at ambient temperature for 6 hours. After this time period the solvent was evaporated, water was added to the residue and acidified to pH 2 with a solution of HCl 1 N, and the aqueous phase was extracted with CH₂Cl₂. The organic phase was dried on anhydrous sodium sulphate and the solvent was evaporated in vacuo. The residue was purified by silica gel chromatography using AcOEt:hexane 5:95 as the eluent to give 790 mg of oily product (yield=71%); TLC: silica gel, eluent AcOEt:hexane 2:8, Frontal ratio (Fr)=0.42; ¹H NMR (CDCl₃, 300 MHz) δ 7.25 (m, 4H), 7.20 (d, 2H), 6.80 (d, 2H), 4.20 (t, 2H), 3.95 (t, 1H), 3.70 (s, 3H), 3.60 (m, 1H), 3.40 (m, 1H), 3.10 (t, 2H), 3.00 (d, 2H), 1.20 (t, 3H); HPLC: column: Inertisil ODS-3 C18 (5 μm) (250×4.6 mm), mobile phase CH₃CN:H₂O (85:15 v/v), pH=as is, T=ambient, flow rate=1 mL/min, 205 nm UV detector, retention time=6.56 min; Elemental Analysis (E.A.) conforms for C₂₀H₂₃ClO₄.

### EXAMPLE 44

### Preparation of dimethyl 4-[2-(2,3-dimethyl-1-indolyl)ethoxy]]benzylmalonate (ST2206)

### Preparation of the intermediate product 2,3-dimethyl-1(2-benzyloxyethyl)indole

To 2,3 dimethyl-1-indole (2.00 g, 13.8 mmol) in anhydrous DMSO (80 mL) were added triturated KOH (1.55 g, 27.6 mmol) and benzyl 2-bromoethylether (5.80 g, 27.6 mmol). The reaction mixture was left at ambient temperature for 20 hours. At the end of this time period H₂O (200 mL) was added to the mixture and the product was extracted with ethyl acetate (3×100 mL). The organic extracts were dried on anhydrous Na₂SO₄ and the solvent was evaporated in vacuo to give 3.20 g of oily product (yield=83%); ¹H NMR (CDCl₃, 300 MHz) δ 7.55 (d, 1H), 7.30-7.10 (m, 8H), 4.42 (s, 2H), 4.30 (t, 2H), 3.80 (t, 2H), 2.40 (s, 3H), 2.30 (s, 3H).

### Preparation of the intermediate product 2,3-dimethyl-1-(2-hydroxyethyl)indole

The product was prepared from 2,3-dimethyl-1-(2-benzyloxyethyl)indole (3.20 g, 11.5 mmol) dissolved in absolute ethanol (100 mL), with 10% Pd/C (800 mg), under H₂ at 50 Psi, at ambient temperature for 4 days. After filtration of the reaction mixture on celite the organic solvent was evaporated in vacuo and the residue purified by silica gel chromatography using hexane:AcOEt 6:4 as the eluent to give 900 mg of product (yield=44%); ¹H NMR (CDCl₃, 300 MHz) δ 7.60 (brd, 1H), 7.30 (d, 1H), 7.15 (m, 2H), 4.30 (t, 2H), 3.95 (t, 2H), 2.40 (s, 3H), 2.30 (s, 3H).

### Preparation of dimethyl 4-[2-(2,3-dimethyl-1-indolyl)ethoxy]benzylmalonate (ST2206)

The product was prepared according to the procedure described in example 14 (method C) starting from dimethyl 4-hydroxybenzylmalonate (1.13 g, 4.76 mmol), prepared as described in example 13, 2,3-dimethyl-1-(2-hydroxyethyl)indole (900 mg, 4.76 mmol), DIAD (1.25 g, 6.2 mmol) and triphenylphosphine (1.62 g, 6.2 mmol), in 90 mL of anhydrous THF, except for the reaction time which was 1 day instead of 5 days and the eluent used in the purification, i.e. hexane:ethyl acetate 7:3 instead of 8:2. The product was further purified by means of two silica gel chomatographies, the first using hexane:ethyl acetate 9:1 and the second CH₂Cl₂ as the eluent to give 506 mg of product (yield=26%); TLC: silica gel, eluent AcOEt:hexane 3:7, Frontal ratio (Fr)=0.50; ¹H NMR (CDCl₃, 300 MHz) δ 7.50 (d, 1H), 7.30 (d, 1H), 7.10 (m, 2H), 7.05 (d, 2H), 6.70 (d, 2H), 4.50 (t, 2H), 4.20 (t, 2H), 3.70 (s, 3H), 3.60 (t, 1H), 3.10 (d, 2H), 2.40 (s, 3H), 2.20 (s, 3H); HPLC: column: Inertisil-ODS-3 (5 μm) (250×4.6 mm), mobile phase CH₃CN:H₂O (80:20 v/v), pH=as is, T=ambient, flow rate=0.9 mL/min, 205 nm UV detector, retention time=9.96 min; Elemental Analysis (E.A.) conforms for C₂₄H₂₇NO₅.

### EXAMPLE 45

### Preparation of (R,S)-2-ethoxy-3-[3-[2-(4-chlorophenyl)ethoxy]phenyl]methyl propanoate (ST 2324)

### Preparation of the intermediate product (Z,E)-2-ethoxy-3-[3-[2-(4-chlorophenyl)ethoxy]phenyl]ethyl propenoate

The product was prepared as described in example 41 (method H) starting from triethyl 2-ethoxyphosphonoacetate (3.6 g, 13.42 mmol), prepared as described in example 41, which was added at 0° C. to a suspension of NaH 80% (480 mg, 15.96 mmol) in anhydrous THF (28 mL), and after approximately 30 minutes at ambient temperature 3-[2-(4-chlorophenyl)ethoxy]benzaldehyde (3.0 g, 11.50 mmol) was added, dissolved in anhydrous THF (20 mL). After evaporation of the solvent in vacuo the residue was purified to give 1.29 g of a mixture of the two isomers (yield=30%); TLC: silica gel, eluent AcOEt:hexane 2:8, Frontal ratio (Fr)=0.32; ¹H NMR (CDCl₃, 300 MHz) δ 7.65 (d, 2H), 7.22 (dd, 4H), 6.95 (s, 1H), 6.85 (d, 2H), 4.30 (q, 2H), 4.20 (t, 2H), 4.00 (q, 2H), 3.10 (t, 2H), 1.40 (t, 6H).

### Preparation of (R,S)-2-ethoxy-3-[3-[2-(4-chlorophenyl)ethoxy]phenyl]methyl propanoate (ST 2324)

To a solution of a mixture of (Z,E)-2-ethoxy-3-[3-[2-(4-chlorophenyl)ethoxy]phenyl]ethyl propenoate (1.29 g, 3.44 mmol) in anhydrous methanol (73 mL) were added Mg in powder form (1.65 g) and a few crystals of I₂, and the mixture was left at ambient temperature for 24 hours. After this time period the solvent was evaporated, water was added to the residue and acidified to pH 2 with a solution of HCl 1 N, and the aqueous phase was extracted with CH₂Cl₂. The organic phase was dried on anhydrous sodium sulphate and the solvent evaporated in vacuo. The residue was purified by silica gel chromatography using AcOEt:hexane 5:95 as the eluent to give 916 mg of oily product (yield=80%); TLC; silica gel, eluent AcOEt:hexane 2:8, Frontal ratio (Fr)=0.45; ¹H NMR (CDCl₃, 300 MHz) δ 7.25-7.20 (m, 5H), 6.80 (m, 3H), 4.15 (t, 2H), 4.00 (t, 1H), 3.70 (s, 3H), 3.60 (m, 1H), 3.35 (m, 1H), 3.05 (t, 2H), 2.95 (d, 2H), 1.15 (t, 3H); HPLC: column: Inertisil ODS-3 C18 (5 μm) (250×4.6 mm), mobile phase CH₃CN:H₂O (85:15 v/v), pH=as is, T=30° C., flow rate=1 mL/min, 205 nm UV detector, retention time=6.42 min; Elemental Analysis (E.A.) conforms for C₂₀H₂₃ClO₄.

### EXAMPLE 46

### Preparation of 5-[3-[2-(4-chlorophenyl)ethoxy]phenylmethylene]thiazolidine-2,4-dione (ST2431)

The product was prepared as described in example 1 (method A) from 3-[2-(4-chlorophenyl)ethoxy]benzaldehyde (1.22 g, 4.70 mmol) in 33 mL of anhydrous toluene, with thiazolidine-2,4-dione (550 mg, 4.70 mmol), acetic acid (37 mg, 0.62 mmol) and piperidine (53 mg, 0.62 mmol) except for the reaction time (5 hours instead of 7 hours). After cooling the mixture, yellow product crystals were separated which were left for 30 minutes at 0° C., then filtered and triturated first with cold toluene and then with water, and then dried. 1.28 g of product were obtained (yield=76%); Melting point (Mp)=186-187° C.; TLC: silica gel, eluent CH₃Cl:CH₃OH 9.8:0.2; Frontal ratio (Fr)=0.45; ¹H NMR (DMSO_{d6}, 300 MHz) δ 12.60 (brs, 1H), 7.70 (s, 1H), 7.40-7.30 (m, 6H), 7.10 (m, 2H), 4.25 (t, 2H), 3.05 (t, 2H); HPLC: column: Symmetry C₁₈ (5 μm) (4.6×150 mm), T=ambient, mobile phase: NH₄H₂PO₄ 0,05 M:CH₃CN (4:6 v/v), pH=as is, flow rate=0.75 mL/min, 205 nm UV detector, retention time 11.25 min; Elemental Analysis (E.A.) conforms for C₁₈H₁₄NO₃SCl

### EXAMPLE 47

### Preparation of 5-[3-[2-(4-chlorophenyl)ethoxy]phenylmethyl]thiazolidine-2,4-dione (ST2390)

To a suspension of ST2431, prepared as described in example 46 (900 mg, 2.50 mmol), in anhydrous MeOH (52 mL), was added piecemeal in small portions Mg in powder form (972 mg, 40.0 mmol). The reaction mixture was left for 5 hours at 25° C. After this time period the solvent was evaporated, water was added to the residue and acidified to pH 2 with a solution of HCl 1 N, and the aqueous phase was extracted with CH₂Cl₂. The pooled organic phases were washed with a saturated solution of NaCl, dried on anhydrous sodium sulphate and evaporated dry in vacuo. The residue thus obtained was purified by silica gel chromatography using CHCl₃ as the eluent to give a product which was still impure that was recrystallised with methanol and then purified again by silica gel chromatography using CHCl₃ as the eluent to give 255 mg of product (yield=28%); Melting point (Mp)=90-91° C.; TLC: silica gel, eluent CHCl₃:CH₃OH 9.8:0.2, Frontal ratio (Fr)=0.45; ¹H NMR (DMSO_{d6}, 300 MHz) δ 12.00 (brs, 1H), 7.40 (m, 5H), 7.20 (t, 1H), 6.80 (m, 3H), 4.90 (dd, 1H), 4.15 (t, 2H), 3.35 (m, 1H), 3.00 (m, 3H); HPLC: column: Symmetry C₁₈ (5 μm) (4.6×250 mm), T=ambient, mobile phase: NH₄H₂PO₄ 0.05M:CH₃CN (4:6 v/v), pH=as is, flow rate 0.7 mL/min, 205 nm UV detector, retention time=12.22 min; Elemental Analysis (E.A.) conforms for C₁₈H₁₆NO₃SCl.

### EXAMPLE 48

### Preparation of dimethyl 3-[[(4-trifluorotolyl)carbamoyl]oxy]benzylmalonate (ST2413)

The product was prepared as described in example 30 (method D) starting from 4-trifluorotolyl isocyanate (1.29 g, 6.93 mmol) and dimethyl 3-hydroxybenzylmalonate, prepared as described in example 22, (1.10 g, 4.62 mmol) in anhydrous THF (30 mL) and NEt₃ (20 μL), except for the fact that the residue obtained after evaporation of the reaction solvent was purified by flash chromatography on silica gel, using AcOEt:hexane 8:2 as the eluent, to give 650 mg of product as a white solid (yield=33%); Melting point (Mp)=93-94° C.; TLC: silica gel, eluent AcOEt:hexane 2:8, Frontal ratio (Fr)=0.13; ¹H NMR (CDCl₃, 300 MHz) δ 7.60 (m, 4H), 7.30 (m, 2H), 7.05 (m, 2H), 3.70 (s+t, 7H), 3.20 (d, 2H); HPLC: column: Symmetry C18 (5 μm) (150×4.6 mm), mobile phase CH₃CN:H₂O (60:40 v/v), pH=as is, T=ambient, flow rate=0.75 mL/min, 205 nm UV detector, retention time=8.77 min; Elemental Analysis (E.A.) conforms for C₂₀H₁₈F₃NO₆.

### EXAMPLE 49

### Preparation of dimethyl 3-[[(2,4-dichlorophenyl)carbamoyl]oxy]benzylmalonate (ST2424)

The product was prepared as described in example 30 (method D) starting from 2,4-dichlorophenylisocyanate (707 mg, 3.78 mmol) and dimethyl 3-hydroxybenzylmalonate, prepared as described in example 22 (600 mg, 2.52 mmol) in anhydrous THF (7 mL), with NEt₃ (10 μL) except for the fact that the residue obtained after evaporation of the reaction solvent was purified by flash chromatography on silica gel, using AcOEt:hexane 2:8 as the eluent, to give 610 mg of product (yield=56.9%); TLC: silica gel, eluent AcOEt:hexane 2:8, Frontal ratio (Fr)=0.40; ¹H NMR (CDCl₃, 300 MHz) δ 8.20 (d, 1H), 7.40 (m, 4H), 7.10 (m, 2H), 3.70 (s+t, 7H), 3.25 (d, 2H); HPLC: column: Symmetry C18 (5 μm)-(150×4.6 mm), mobile phase CH₃CN:H₂O (60:40 v/v), pH=as is, T=ambient, flow rate=0.75 mL/min, 205 nm UV detector, retention time=9.51 min; Elemental Analysis (E.A.) conforms for C₁₉H₁₇Cl₂NO₆.

The compounds according to the invention described herein are useful as medicines, particularly for the preparation of medicines with serum glucose and serum lipid lowering activity. The preferred applications are the prophylaxis and treatment of diabetes, particularly type 2, and its complications, Syndrome X, the various forms of insulin resistance and hyperlipdaemias.

In a thoroughly advantageous manner, the compounds according to the invention described herein are endowed with good pharmacological activity, but present reduced liver toxicity.

Experiments have been conducted in vivo in diabetic mouse models and in vitro in adipocyte 3T3-L1 cell lines (reported in the literature in predictive assays for potential antidiabetic activity—see, for example, Sarges et al., J Med Chem 39: 4783-4803, 1996, Luo et al., Diabetic Med 15: 367-374, 1998 and Bierer et al., J Med Chem 41: 894-901, 1998).

Pharmacological Activity

Determination of Glucose Consumption in 3T3-L1 Cells

Glucose consumption was assessed in differentiated 3T3-L1 cells.

Mouse fibroblasts (3T3-L1) were seeded at a density of 5×10³/cm² and cultured on 12-well plates in 1 ml of DMEM containing glucose 25 mM and added with 10% CS, glutamine 4 mM, pyruvate 1 mM, penicillin 50 U/ml, and streptomycin 50 μg/ml, in an atmosphere humidified with 5% CO₂ at 37° C.

Two to three days after confluence, differentiation was induced with the addition of 1.5 ml of DMEM containing 3-isobutyl-1-methylxanthine (IBMX) 0.5 mM, dexamethazone 1 μM and porcine insulin 10 μg/ml in glucose 25 mM and 10% FBS.

After 2 days, the cells were exposed to the same medium without IBMX and dexamethazone for another 2 days.

The cells were then maintained in DMEM containing glucose 25 mM and 10% FBS over the next few days, with changes of culture medium at intervals of 2-3 days (Clancy B M and Czech M P, J. Biol. Chem., 265: 12434-12443, 1990; Frost S C and Lane M. D, J. Biol. Chem. 260: 2645-2652, 1985).

The cells were used 10-12 days after induction of differentiation, as monitored by evaluating triglyceride accumulation.

For the assessment of glucose consumption, the cells were incubated for 22 hours in DMEM containing glucose 25 mM, insulin 0.25 nM (submaximal concentration) and the compounds (1, 5, 10, 25 μM) dissolved in DMSO (final concentration 0.1%).

Rosiglitazone was used as a positive control.

The analysis of the glucose in the medium was done with the aid of a Cobas Mira S autoanalyzer (Roche), using the HK 125 Glucose Kit (ABX Diagnostics). The glucose concumprion stimulated by the products was evaluated as % increase compared to the control compound.

Taking compound 22 as an example, Table 1 gives the lowest concentration of those assayed to induce a 40% increase in glucose consumption compared to the control compound (rosiglitazone).

From the results obtained it can be deduced that the compounds investigated were capable of increasing glucose consumption in 3T3-L1 cells to a similar extent to that achieved by the reference compound (rosiglitazone).

TABLE 1

| Compound | μM* |
|---|---|
| Rosiglitazone | 5 |
| Example 22 | 1 |

Antidiabetic and Serum Lipid Lowering Activity in db/db Mice

Mutations in laboratory animals have made it possible to develop models that present non-insulin-dependent diabetes associated with obesity, hyperlipidaemia and insulin-resistance and that enable us to test the efficacy of new antidiabetes compounds (Reed and Scribner, Diabetes, obesity and metabolism 1: 75-86, 1999).

A genetically diabetic mouse model much used by the pharmaceutical companies is the C57BL/KsJ db/db mouse.

The genetic basis of this model is a defect in the leptin receptor gene, which causes leptin resistance and leads to hyperphagia, obesity, hyperinsulinaemia and insulin resistance, with subsequent symptoms of insufficient insular secretion and hyperglycaemia (Kodama et al., Diabetologia 37: 739-744, 1994; Chen et al., Cell 84: 491-495, 1996).

Since hyperglycaemia is accompanied by obesity and insulin resistance, the db/db mouse has characteristics that resemble those of type 2 diabetes in man and is useful for assaying insulin-sensitising compounds.

The thiazolidinediones constitute one class of such compounds (Day, Diabet. Med. 16: 179-192, 1999; Mudaliar and Herry, Annu. Rev. Mred. 52: 239-257, 2001, Drexler et al., Geriatrix 56: 20-33, 2001).

Of the three thiazolidinediones launched on the market, troglitazone was withdrawn owing to its severe liver toxicity, while the other two compounds, rosiglitazone and pioglitazone, which are effective in reducing diabetic hyperglycaemia, are known to present weight gain, oedema, liver toxicity, increased LDL-cholesterol, and anaemia as side effects (Schoonjans and Auwerx, The Lancet 355: 1008-1010, 2000; Peters, Am. J. Manag. Care 7: 587-595, 2001; Gale, The Lancet 357: 1870-1875, 2001).

The C57BL/KsJ db/db mice in the experiments were supplied by Jackson Lab (via Ch. River). After 10 days of acclimatisation in standard conditions (22±2° C.; 55±15% humidity; 15-20 air changes/hour; 12 hour light-dark cycle, with light from 7.00 a.m to 7.00 p.m), and on a standard 4 RF21 diet (Mucedola), blood samples were taken in postabsorption conditions (fasting from 8.30 a.m to 4.30 p.m.) from the caudal vein with the aid of a Jelco 22G catheter (Johnson and Johnson). Plasma levels of glucose, insulin, triglycerides, cholesterol, free fatty acids and urea were monitored to ensure a well-matched distribution of the mice in the treatment groups.

At the start of treatment, the animals' body weights were checked and arrangements were made for monitoring water and feed consumption.

The mice were treated orally twice daily (8.30 a.m. and 6.30 p.m.) for a fortnight.

The compounds were administered at a dose equivalent to 25 mg/kg of the compound in example 22 in 10 ml/kg of vehicle (CMC 1% containing Tween 80 0.5% in deionised H₂O). Rosiglitazone was administered at the dose of 5 mg/kg (Lohray et al. J. Med Chem 41, 1619-1630, 1998).

The animals were sacrificed (by decapitation) in postabsorption conditions (fasting from 9.30 a.m. to 4.30 p.m.) 7 hours after the last treatment. Serum levels of a number of important lipid and carbohydrate metabolism variables were measured.

The compounds according to the invention described herein show a good ability to reduce serum triglyceride levels in a manner similar to the reference compound rosiglitazone. Table 2, by way of an example, shows the serum lipid lowering activity of the compound in example 22 and of rosiglitazone.

The compounds, moreover, are, like rosiglitazone, also capable of lowering serum glucose levels (Table 3) and this is achieved with lesser changes in weight and transaminase (GPT) values, which is indicative of less liver damage (Table 4). By way of an example, Table 3 gives the serum glucose lowering activity of the example 22 compound and Table 4 the changes in weight and transaminase values in the same compound, again as compared to rosiglitazone. Furthermore, unlike rosiglitazone, the compounds according to the invention increase HDL-cholesterol levels. By way of an example, Table 4 gives the changes in HDL-cholesterol levels for the compound in example 22 and for the reference compound rosiglitazone. An increase in HDL-cholesterol constitutes an indicator of PPARα agonism and of a reduced risk of atherosclerosis. PPARα agonism, in fact, increases fatty acid oxidation in the tissues, reducing the accumulation of intracellular triglycerides, which favour insulin resistance (Virkamaki et al., Diabetes 50, 2337-2343, 2001; Mensink et al., Diabetes 50, 2545-2554, 2001; Kelley and Goodpaster, Diabetes Care 24, 933-941, 2001). It is known, for example, that the fibrates, which are PPARα agonists, not only lower hyperlipidaemia, but are also capable of improving insulin sensitivity (Matsui et al., Diabetes 46, 348-353, 1997), atherosclerosis and cardiovascular damage (Fruchart et al., Current Atherosclerosis Reports 3, 83-92, 2001), which is a serious complication and cause of death in the course of diabetic disease.

The usefulness of these compounds for correcting hyperlipidaemia, diabetes and the cardiovascular complications accompanying these disease conditions is evident.

TABLE 2
Serum lipid lowering activity in db/db mice

| | Dose | Reduction of |
|---|---|---|
| Compound | (mg/kg) | triglyceride levels % |
| Rosiglitazone | 5 | −41 ▴ |
| Example 22 | 25 | −47 ▴ |

Student's ‘t’-test: ▴ indicates P < 0.001 vs control.

TABLE 3
Serum glucose lowering activity in db/db mice

| | Dose | Reduction of |
|---|---|---|
| Compound | (mg/kg) | glucose levels % |
| Rosiglitazone | 5 | −36 Δ |
| Example 22 | 25 | −32 Δ |

Student's ‘t’-test: Δ indicates P < 0.01 vs control

TABLE 4
Weight gain and changes in GPT and HDL-cholesterol
serum levels in db/db mice

| | | | | Change in |
|---|---|---|---|---|
| | Dose | Weight | Change in | HDL-cholesterol |
| Compound | (mg/kg) | gain % | GPT levels % | levels % |
| Rosiglitazone | 5 | +22 ▴ | +117 ▴ | −7 |
| Example 22 | 25 | +16 ▴ | +38 ▴ | +37 ▴ |

Student's ‘t’-test: ▴ indicates P < 0.001 vs control.

The subject of the invention described herein are pharmaceutical compositions containing as their active ingredient at least one formula (I) compound, or, said formula (I) compound or compounds in combination with other active ingredients useful in the treatment of the diseases indicated in the invention described herein, e.g. other products endowed with serum glucose and serum lipid lowering activity, also in separate dosage form or in forms is suitable for combined therapies. The active principle according to the invention described herein will be in a mixture with suitable vehicles and/or excipients commonly used in pharmacy, such as, for instance, those described in “Remington's Pharmaceutical Sciences Handbook”, latest edition. The compositions according to the invention described herein will contain a therapeutically effective amount of the active ingredient. The dosages will be determined by the expert in the sector, e.g. the clinican or primary care physician, according to the type of disease to be treated and the patient's condition, or concomitantly with the administration of other active ingredients. By way of an example we may indicate dosages ranging from 0.1 to 200 mg/day.

Examples of pharmaceutical compositions are those that permit oral or parenteral, intravenous, intramuscular, subcutaneous and transdermal administration. Suitable pharmaceutical compositions for this purpose are tablets, rigid or soft capsules, powders, solutions, suspensions, syrups, and solid forms for extempore liquid preparations. Compositions for parenteral administration are, for example, all the intramuscular, intravenous and subcutaneous injectable forms, in the form of solutions, suspensions and emuslions. Liposomal formulations should also be mentioned. Also included are the forms characterised by controlled release of the active ingredient, whether as oral administration forms, tablets coated with suitable layers, microencapsulated powders, complexes with cyclodextrin, or depot forms, e.g. of the subcutaneous type, such as depot injections or implants.

1. Formula (I) compounds: 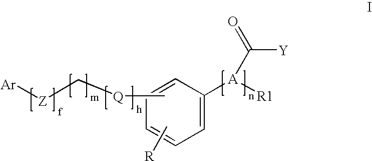
where:
A is CX; alkanylilidene with 2 to 4 carbon atoms, particularly CH₂—CH; alkenylilidene with 2 to 4 carbon atoms, particularly CH═C;
Ar is monocyclic or bicyclic C₆-C₁₀ aryl or heteroaryl, containing one or more heteroatoms selected from the group consisting of nitrogen, oxygen and sulphur, possibly substituted by halogens, NO₂, OH, C₁-C₄ alkyl and alkoxy, said alkyl and alkoxy possibly substituted by at least one halogen; monocyclic, bicyclic or tricyclic arylalkyl or heteroarylalkyl containing one or more heteroatoms selected from the group consisting of nitrogen, oxygen and sulphur, where the alkyl residue contains from 1 to 3 carbon atoms, said arylalkyl or heteroarylalkyl possibly substituted by halogens, NO₂, OH, C₁-C₄ alkyl and alkoxy,
said alkyl and alkoxy possibly substituted by at least one halogen;
f is the number 0 or 1;
h is the number 0 or 1;
m is a whole number from 0 to 3;
n is the number 0 or 1 and if n is O, R₁ is absent, and COY is directly bound to benzene);
Q and Z, which may be the same or different, are selected from the group consisting of NH, O, S, NHC(O)O, NHC(O)NH, NHC(O)S, OC(O)NH, S(CO)NH, C(O)NH, and NHC(O);
R is selected from R₂, OR₂;
R₁ is selected from H, COW, SO₃-, OR₃, =0, CN, NH₂, NHCO(C₆-C₁₀)Ar, where Ar may possibly be substituted by halogens, NO₂, OH, C₁-C₄ alkyl and alkoxy, said alkyl and alkoxy possibly substituted by at least one halogen;
R₂ is selected from H, straight or branched C₁-C₄ alkyl, possibly substituted by at least one halogen;
R₃ is selected from H, straight or branched C₁-C₄ alkyl, possibly substituted by at least one halogen, (C₆-C₁₀)ArCH₂, where Ar is possibly substituted by halogens, NO₂, OH, C₁-C₄ alkyl and alkoxy, said alkyl and alkoxy possibly substituted by at least one halogen;
W is selected from OH, OR₄, NH₂;
R₄ is straight or branched C₁-C₄ alkyl;
Y is selected from OH, OR₅, NH₂;
R₅ is straight or branched C₁-C₄ alkyl;
or A, COY and R1 together form a cycle of the type: 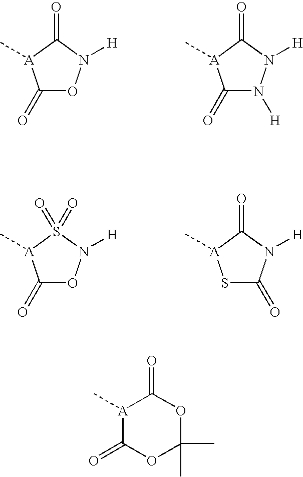
their pharmacologically acceptable salts, racemic mixtures, individual enantiomers, geometric isomers or stereoisomers, and tautomers.

2. Compounds according to claim 1, in which Ar is a heteroaryl, preferably containing nitrogen as the heteroatom, and preferably f is 0, m is 1 or 2, Q is oxygen, and R is hydrogen.

3. Compounds according to claim 1, in which Ar is an aryl, possibly substituted by one or more halogen atoms, alkyl, alkoxy or lower haloalkyl, nitro, mono- or di-alkylamine, and preferably f is 0, m is 0, 1 or 2, Q is oxygen or HNC(O)O, and R is hydrogen.

4. Compounds according to claim 1, where R₁ is COW.

5. Compound according to claim 1, selected from the group consisting of:
i. Diethyl 4-[2-(1-indolyl)ethoxy]benzylidenemalonate;
ii. Diethyl 4-[2-(1-indolyl)ethoxy˜benzylmalonate;
iii. Dimethyl 4-[2-(1-indolyl)ethoxy]benzylidenemalonate;
iv. Dimethyl 4-[2-(1-indolyl)ethoxy]benzylmalonate;
V. 4-[2-(1-indolyl)ethoxy]benzylmalonic acid;
vi. Methyl (2S)-amino-2-[4-[2-(1-indolyl)ethoxy]phenyl]-acetate;
vii. Methyl 4-[2-(1-indolyl)ethoxy]benzoate;
viii. Methyl 3-[4-[2-(1-indolyl)ethoxy]phenyl]propanoate;
ix. Methyl 2-[4-[2-(1-indolyl)ethoxy]phenyl]acetate;
x. Methyl 2-sulpho-2-[4-[2-(1-indolyl)ethoxy]phenyl]acetate sodium salt;
xi. Methyl (S)-2-benzoylamino-2-[4-[2-(1-indolyl)ethoxy]-phenyl] acetate;
xii. Methyl 2-hydroxy-3-[4-[2-(1-indolyl)ethoxy]phenyl]-propanoate;
xiii. Dimethyl 4-[2-[4-(dimethylamino)phenyl]ethoxy]benzylmalonate;
xiv. Methyl 3-[4-[2-(1-indolyl)ethoxy]phenyl]-2-cyano-propenoate;
xv. Methyl 3-[4-[2-(1-indolyl)ethoxy]phenyl]-2-cyano-propanoate;
xvi. Dimethyl 4-[2-(3-indolyl)ethoxy]benzylidenemalonate;
xvii. Dimethyl 4-[2-(1-naphthyl)ethoxy]benzylmalonate;
xviii. Dimethyl 4-[2-(2-pyridyl)ethoxy]benzylmalonate;
xix. Dimethyl 4-[2-(4-chlorophenyl)ethoxy]benzylmalonate;
xx. 5-[4-[2-(4-chlorophenyl)ethoxy]phenylmethylene]-thiazolidine-2,4-dione;
xxi. 5-[4-[2-(4-chlorophenyl)ethoxy]phenylmethyl]thiazolidine-2,4-dione;
xxii. Dimethyl 3-[2-(4-chlorophenyl)ethoxy]benzylmalonate;
xxiii. Dimethyl 3-[2-(phenyl)ethoxy]benzylmalonate;
xxiv. Dimethyl 3-[N-(4-trifluoromethylbenzyl)carbamoyl]-4-methoxybenzylmalonate;
xxv. Dimethyl 4-methoxy-3-[2-(4-chlorophenyl)ethoxy]benzyl-malonate:
xxvi. Dimethyl 3-(2-phenylethoxy)-4-methoxy benzylmalonate;
xxvii. Dimethyl 4-[2-(4-methoxyphenyl)ethoxy]benzylmalonate;
xxviii. Dimethyl 4-[3(4-methoxyphenyl)propyloxy]benzyl-malonate;
xxix. Dimethyl 4-[2-(2-naphthyl)ethoxy]benzylmalonate;
xxx. (2S)-2-benzoylamino-3-[4-[(4-methoxybenzyl)-carbamoyl-]oxypheny]ethyl propanoate;
xxxi. Dimethyl 4-[[(4-methoxybenzyl)carbamoyl]oxy]benzyl-malonate;
xxxii. Dimethyl 4-[[(4-trifluorotolyl)carbamoyl]oxy]benzyl-malonate;
xxxiii. Dimethyl 4-[[(2,4-dichlorophenyl)carbamoyl]oxy]benzyl-malonate;
xxxiv. Dimethyl 4-[[(4-chlorophenyl)carbamoyl]oxy]benzyl-malonate;
xxxv. Dimethyl 4-[2-(pyridinio)ethoxy]benzylmalonate methanesulphonate;
xxxvi. Dimethyl 4-[[(4-nitrophenyl)carbamoyl]oxy]benzyl-malonate;
xxxvii. Dimethyl 3-[[(4-methoxybenzyl)carbamoyl]oxy]benzylmalonate;
xxxviii. Dimethyl 3-[[(4-butylphenyl)carbamoyl]oxy]benzyl-malonate;
xxxix. Dimethyl 4-[[(4-butylphenyl)carbamoyl]oxy]benzyl-malonate;
xl. Dimethyl 3-[[(4-chlorophenyl)carbamoyl]oxy]benzyl-malonate;
xli. (Z)-2-ethoxy-3-[4-[2-(4-chloro-phenyl)ethoxy]-phenyl]ethyl propenoate;
xlii. (E)-2-ethoxy-3-[4-[2-(4-chloro-phenyl)ethoxy]-phenyl]ethyl propenoate;
xliii. (R,S)-2-ethoxy-3-[4-[2-(phenyl)ethoxy]phenyl]ethyl propanoate;
xliv. (R,S)-2-ethoxy-3-[4-[2-(4-chloro-phenyl)ethoxy]-phenyl-]methyl propanoate;
xlv. Dimethyl 4-[2-(2,3-dimethyl-1-indolyl)ethoxy]benzyl-malonate.

6. Compounds according to claim 1 as medicines.

7. Pharmaceutical compositions containing at least one compound according to claim 1 in mixtures with pharmaceutically acceptable vehicles and! or excipients.

8. Use of the compounds according to claim 1 for the preparation of a medicine with serum glucose and serum lipid lowering activity.

9. Use of the compounds according to claim 1 for the preparation of a medicine for the prophylaxis and treatment of diabetes, particularly type 2, and its complications, Syndrome X, the various forms of insulin resistance and hyperlipdaemias.
